(19) United States
(12) Patent Application Publication
(10) Pub. No.: US 2024/0124461 A1
(43) Pub. Date: Apr. 18, 2024

(54) PROCESSES FOR THE PREPARATION OF ZANUBRUTINIB AND INTERMEDIATES THEREOF

(71) Applicant: TEVA PHARMACEUTICALS INTERNATIONAL GMBH, Jona (CH)

(72) Inventors: Tihamér Paál, Debrecen (HU); Zsuzsa Potarine Juhasz, Debrecen (HU); Tomislav Biljan, Krizevci (HR); Irena Nekola, Sisak (HR); Moris Mihovilovic, Zagreb (HR)

(21) Appl. No.: 18/038,506

(22) PCT Filed: Dec. 10, 2021
(86) PCT No.: PCT/US21/62755

§ 371 Date: May 24, 2023

Related U.S. Application Data
(60) Provisional application No. 63/124,137, filed on Dec. 11, 2020.

(51) Int. Cl. C07D 487/04

(52) U.S. Cl. CPC C07D 487/04; C07B 2200/13

(57) ABSTRACT

The present disclosure provides new procedures and intermediates for the preparation of Zanubrutinib.

### FIELD OF THE INVENTION

The present disclosure provides new procedures and intermediates for the preparation of Zanubrutinib.

### BACKGROUND OF THE INVENTION

Zanubrutinib has the chemical name (7S)-2-(4-phenoxyphenyl)-7[1-(prop-2-enoyl)piperidin-4-yl]-4,5,6,7-tetrahydropyrazolo[1,5-a]pyrimidine-3-carboxamide, or alternatively (7S)-4,5, 6,7-Tetrahydro-7-[1-(1-oxo-2-propen-1-yl)-4-pperidinyl]-2-(4-phenoxyphenyl)pyrazolo[1,5-a]pyrimidine-3-carboxamide. Zanubrutinib has the following chemical structure:

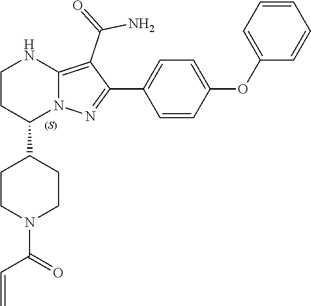

Zanubrutinib is a Bruton's tyrosine kinase (BTK) inhibitor, marketed in the US as Brukinsa® by BeiGene USA Inc., as an orally administered treatment for adult patients with mantle cell lymphoma (MCL) who have received at least one prior therapy. Zanubrutinib is also under investigation for the treatment of B-cell malignancies, relapsed/refractory chronic lymphocytic leukemia, small lymphocytic lymphoma, treatment-naïve 17p-deletion chronic lymphocytic leukemia, relapsed refractory marginal zone lymphoma, and Waldenström's macroglobulinemia.

Zanubrutinib is disclosed in International Publication No. WO 2014/173289, U.S. Pat. No. 9,447,106 and EP Patent No. 2989196B. Crystalline forms of Zanubrutinib are disclosed in International Publication No. WO 2018/033853.

Processes for preparation of Zanubrutinib and/or the intermediates are described in International Publication No. WO 2018/033853 and CN Patent No. 110845504A.

U.S. Pat. No. 9,447,106 (US '106) describes a process for the preparation of Zanubrutinib, which involves a final step of chiral HPLC separation of racemic Zanubrutinib:

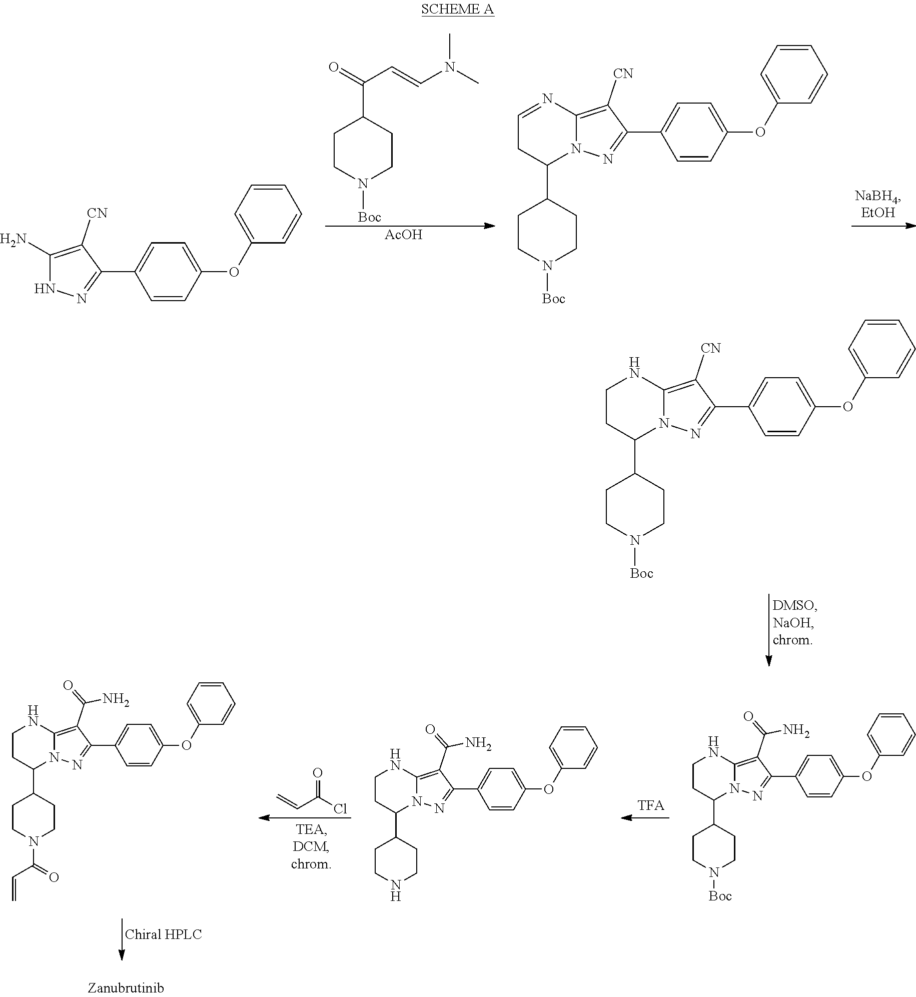

A process for preparing Zanubrutinib is also disclosed in International Publication No. WO 2018/033853 (WO '853). The process involves the same initial coupling reaction as described in US '106, but chiral resolution is carried out three times over different process steps:

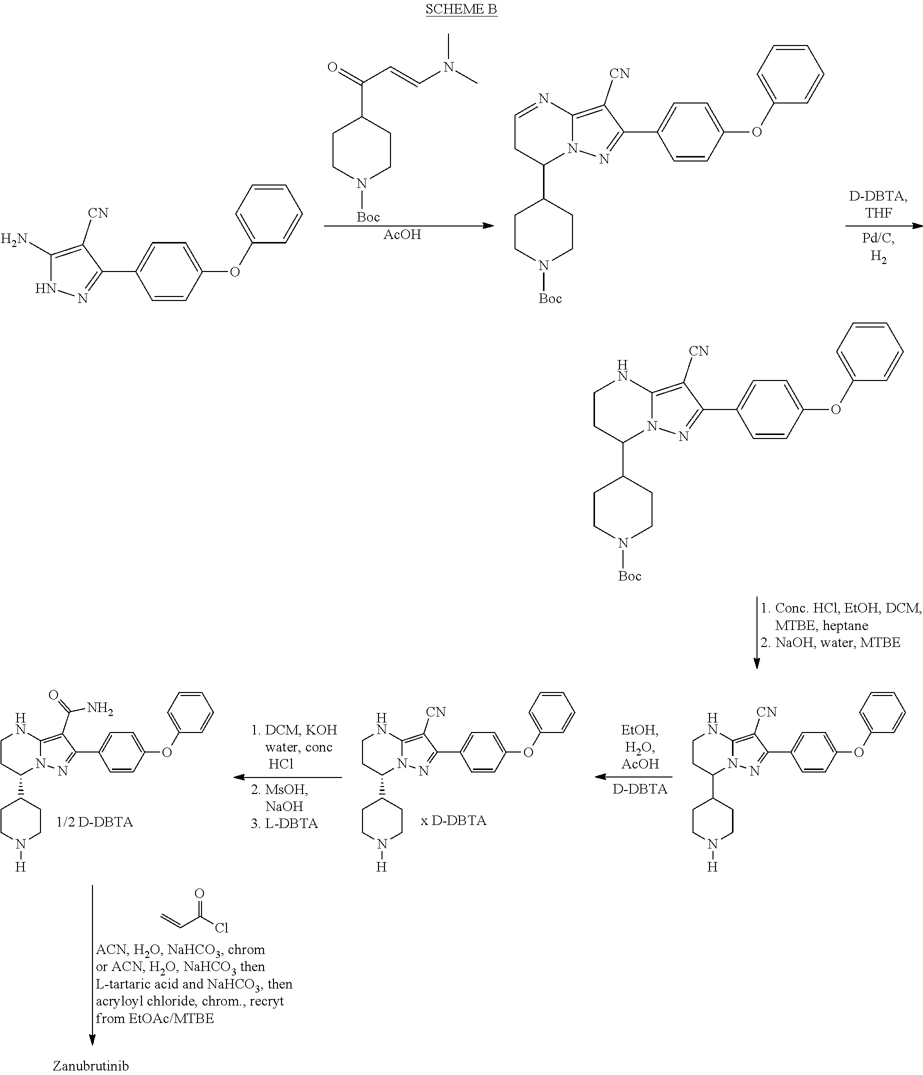

The synthetic process disclosed in CN Patent No. 110845504 (“CN 504”) patent involves conversion of the nitrile group to amide, followed by reaction with acryloyl chloride. These transformations are disclosed in WO '853, and also, in relation to the racemic compounds in US '106:

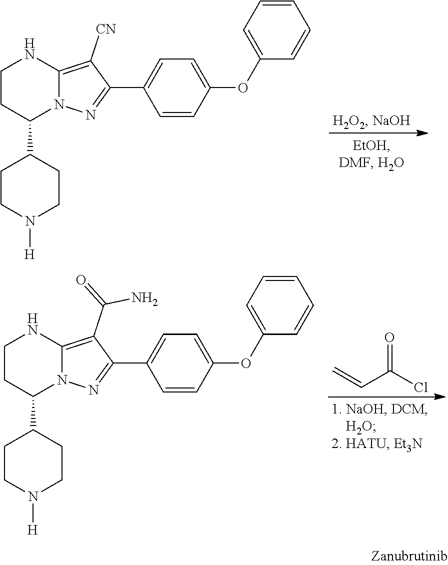

According to CN '504, column chromatography is used in order to purify the intermediate and the final Zanubrutinib product.

There is a need in the art for improved processes for synthesizing Zanubrutinib or any salt thereof, particularly with high yields, high chemical purity and high optical purity, which are suitable for industrial use. Particularly, it would be desirable to avoid the need for multiple chiral purification steps and multiple chromatographic purification steps. Moreover, the use of chiral HPLC in the final step, as disclosed in US '106 is highly undesirable and is not suitable for application to an industrial-scale synthesis.

### SUMMARY OF THE INVENTION

The present disclosure provides processes for the preparation of Zanubrutinib or any salt thereof, as well as novel intermediates, their preparation and their use in the preparation of Zanubrutinib or any salt thereof.

The present disclosure provides a process for preparing Zanubrutinib, or any salt thereof, comprising a step of reacting a compound (IX) with compound (VIII) to obtain compound (VII):

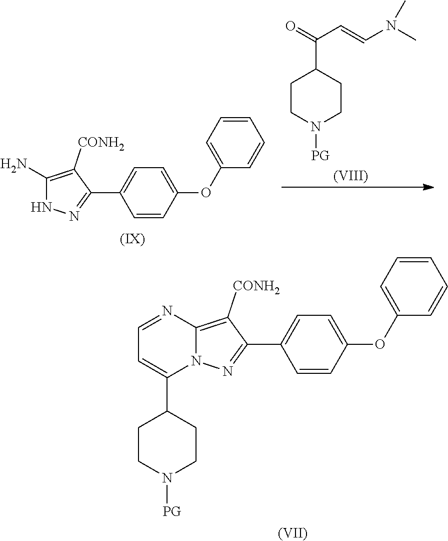

wherein PG is a protecting group; and converting the compound of formula (VII) to Zanubrutinib or a salt thereof.

The compound (VII) can be reduced and deprotected, sequentially in any order, or simultaneously, to obtain a compound of formula (VI):

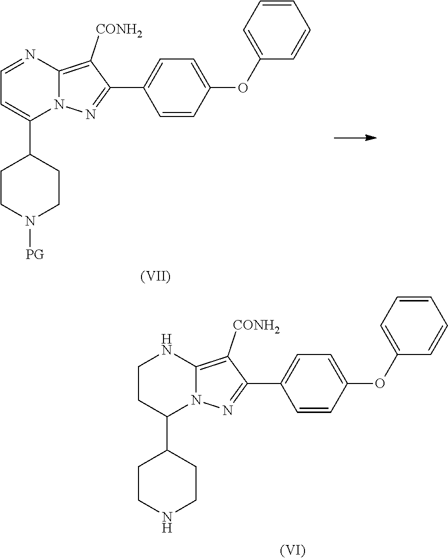

In any of the processes of the present disclosure, the compound of formula (IX) may be prepared by a process comprising basification of an acid addition salt of the compound of formula (X):

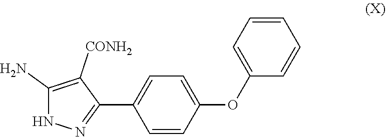 [SMILES: NC(=O)c1c(-c2ccc(Oc3ccccc3)cc2)n[nH]c1N]

wherein the salt is with methane sulfonic acid, p-toluene sulfonic acid or polyphosphoric acid.

The disclosure further provides a method of converting the compound of formula (VII) to Zanubrutinib, comprising the step of:
(a) subjecting the compound (VII) to reduction and deprotection sequentially in any order, or simultaneously, to form a compound (VI):

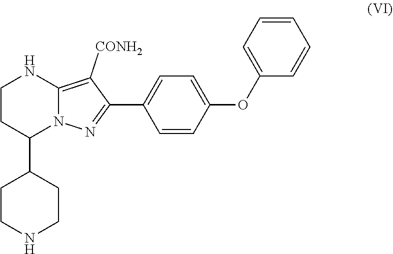 [SMILES: NC(=O)c1c(-c2ccc(Oc3ccccc3)cc2)nn2c1NCCC2C1CCNCC1]

b) chiral resolution of the compound (VI) to form a compound (III):

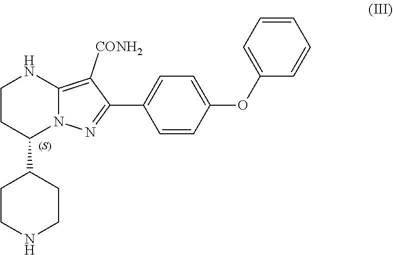

c) N-substitution of the compound (III) to form Zanubrutinib (I):

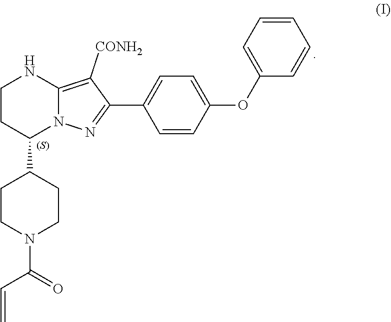

The present disclosure further provides a process for preparing Zanubrutinib comprising the steps of:
(A) reacting a compound (IX) with a compound (VIII) to form the compound (VII):

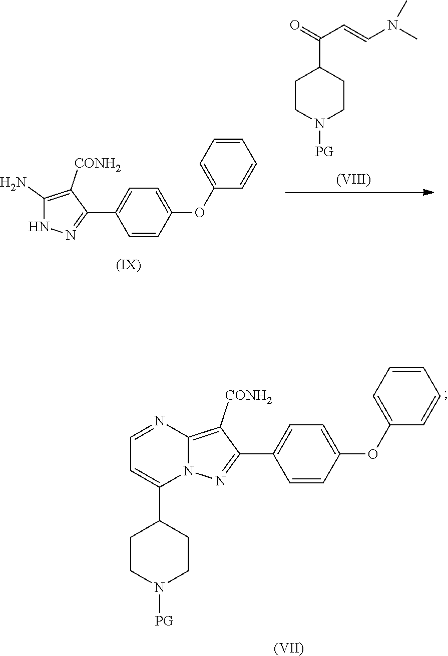

(B) reducing and deprotecting the compound (VII) to form a compound (VI):

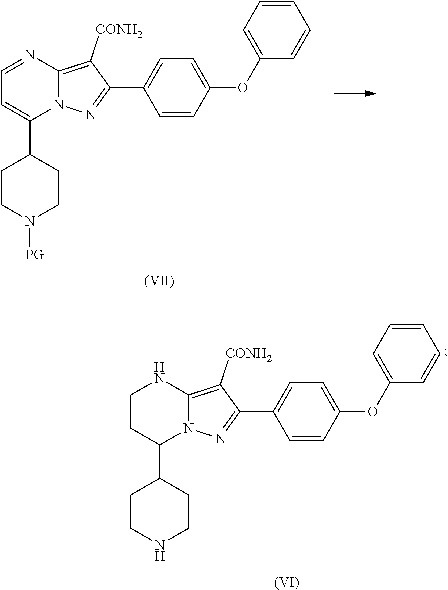

(C) chiral resolution of the compound (VI) to form a compound (III):

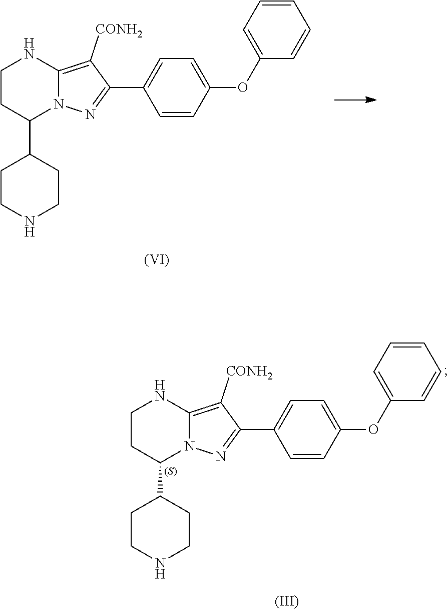

and
(D) N-substitution of the compound (III):

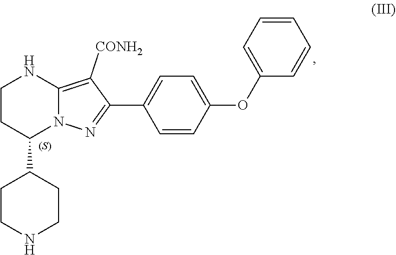

to form Zanubrutinib.

The process steps (A), (B), (C), and (D) may be carried out according to any of the herein described processes.

Also disclosed are compounds selected from formula (VII) and formula (II), as described below:

### Formula (VII):

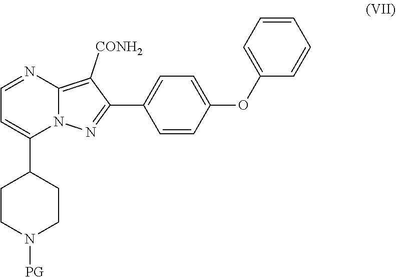

wherein PG is a protecting group, optionally acetyl, benzyl, methyl, benzoyl, toluoyl, methoxycarbonyl, ethoxycarbonyl, benzyloxycarbonyl, tert-butyloxycarbonyl, allyloxycarbonyl, 4-methoxybenzyl, para-methoxybenzylcarbonyl, 3,4-dimethoxybenzyoyl, propionyl, butyryl, phenylacetyl, phenoxyacetyl, trityl, 2,2,2-trichloroethoxycarbonyl, carbobenzoxy, 4-methoxybenzyloxycarbonyl, 9-fluorenylmethoxycarbonyl, 2-iodoethoxycarbonyl, 4-methoxy-2,3,6-trimethylbenzenesulfonyl, methanesulfonyl, para-toluenesulfonyl, phenyl sulfonyl, trifluorocarbonyl, 2-trimethyl silylethoxycarbonyl, 4-nitrobenzenesulfonyl; and

### Formula (II):

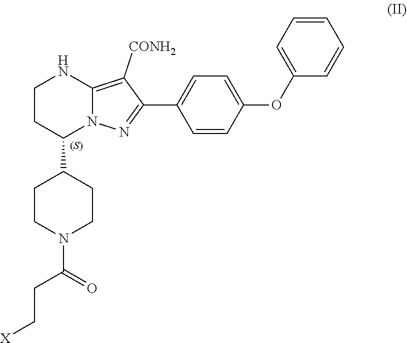 wherein X is chloro or bromo.

The compounds of Formula (V), (VII) and (II) as described herein are useful intermediates of formula (II) that can be advantageously used in the preparation of Zanubrutinib.

The present disclosure further provides isolated compounds of formula, (VII) and (II). Also provided are compounds of formula (VII) and (II) in solid state form.

The disclosure further provides the use of a compound of Formula (VII), or (II) in the preparation of Zanubrutinib, or any salt thereof

According to any aspect or embodiment of the present disclosure, the compounds (VII) or (II) may be substantially pure, i.e. having a purity of: greater than about 95%, greater than about 97%, greater than about 99%, or greater than about 99.3%, or greater than about 99.8% (by weight).

In yet a further aspect, the disclosure provides processes for preparing Zanubrutinib, comprising preparing compound of formula (V), (VII) or (II) and converting the compound to Zanubrutinib or any salt thereof by any method disclosed herein.

In a still further aspect, the disclosure provides an alternative process for preparing Zanubrutinib or a salt thereof, comprising a step of reacting a compound (IX) as described above, with a compound (XII) to obtain compound (XIII):

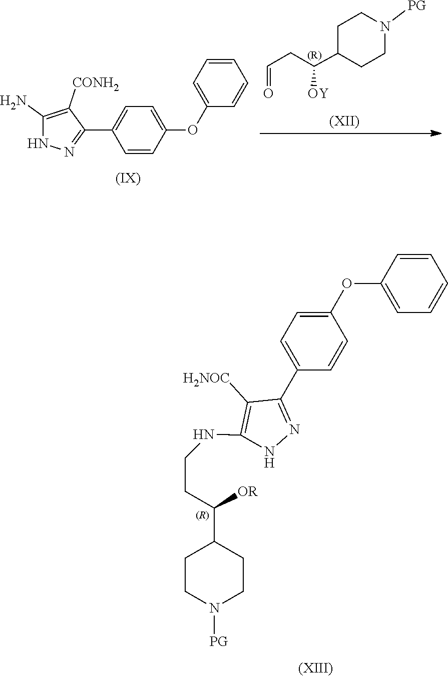

wherein PG is a protecting group as defined according to any embodiment of the disclosure, and R represents H or a protecting group P I, which is optionally trialkylsilyl.

In a still further aspect, the disclosure provides a process for preparing a compound (XII) as described above. The process may be represented by the following scheme:

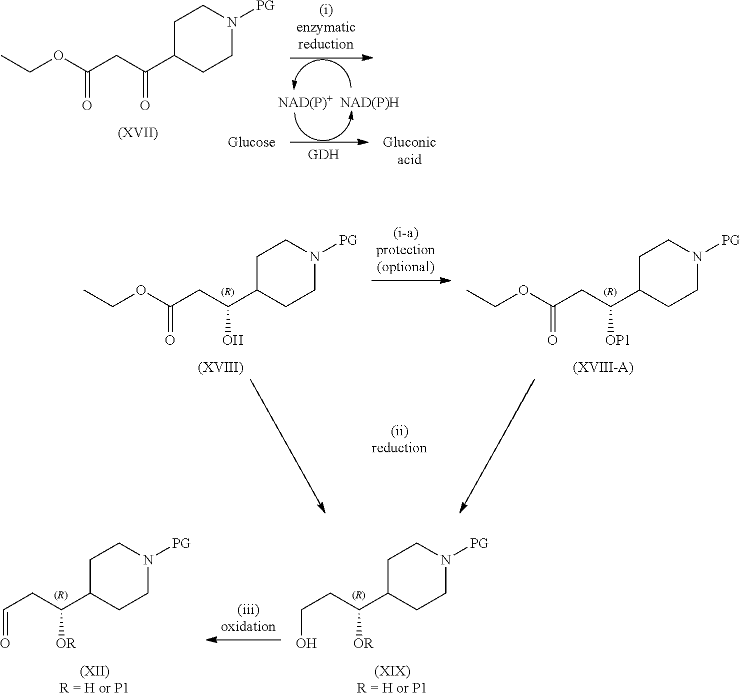

(i) biocatalytic (enzymatic) reduction of a compound of formula (XVII) to form a compound of formula (XVIII) [step (i)];
(ii) optionally protecting the hydroxy group of the compound of formula (XVIII) form a compound of formula (XVIII-A) [step (i-a)], and
(iii) reduction of the compound (XVIII) or (XVIII-A) to form a compound of formula
(XIX) wherein R=H or P1, respectively [step (ii); and
(iv) oxidation of the compound of formula (XIX) to from the compound of formula
(XII) [step (iii)];
   wherein PG is a protecting group as defined according to any of the disclosure herein; P1 is a protecting group, optionally trialkylsilyl.

In yet a further aspect, the compound of formula (XIII) may be converted to Zanubrutinib by a process comprising:
cyclisation of the compound (XIII) to form a compound (XIV):

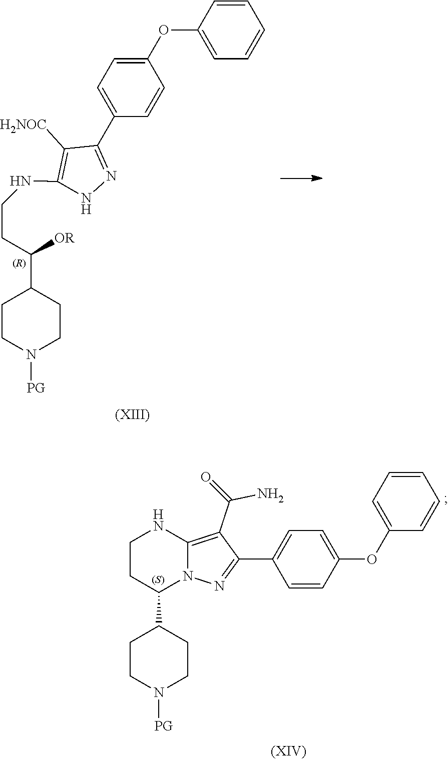

deprotection of the compound (XIV) to form the compound (III):

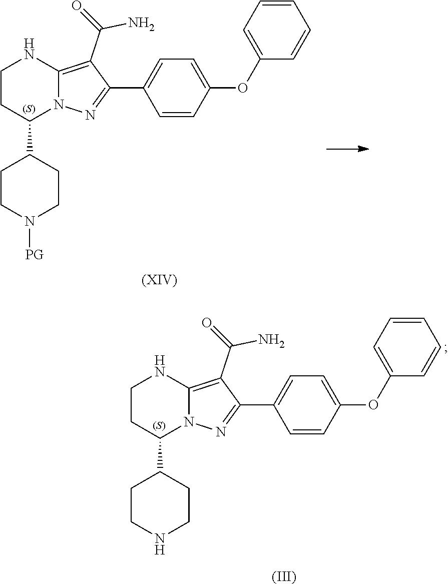

N-substitution of the compound of formula (III):

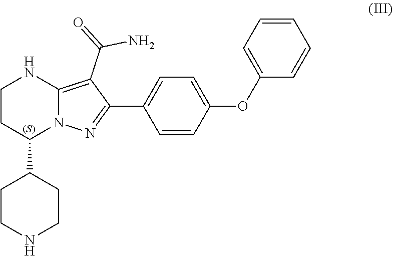

to form Zanubrutinib.

The present disclosure further provides compounds of formula (XIII) and (XII), and their use in the synthesis of Zanubrutinib or a salt thereof.

In yet a further aspect, the disclosure provides Zanubrutinib or any salt thereof prepared by the processes of the disclosure.

In a further aspect, Zanubrutinib or any salt thereof prepared according to present disclosure is substantially pure.

In yet a further aspect, Zanubrutinib or any salt thereof prepared according to present disclosure is substantially free of impurities.

A further aspect of the disclosure provides a process as described herein, further comprising combining the Zanubrutinib or a salt thereof with at least one pharmaceutically acceptable excipient to form a pharmaceutical formulation, optionally wherein the pharmaceutical formulation is an oral dosage form.

The present disclosure also provides use of the Zanubrutinib prepared according to present process for treating mantle cell lymphoma (MCL) in adults who have received at least one prior therapy, B-cell malignancies, relapsed/refractory chronic lymphocytic leukemia, small lymphocytic lymphoma, treatment-naïve 17p-deletion chronic lymphocytic leukemia, relapsed refractory marginal zone lymphoma, and Waldenström's macroglobulinemia.

### BRIEF DESCRIPTION OF THE FIGURES

FIG. 1: Powder XRD diffractogram of crystalline 5-amino-3-(4-phenoxyphenyl)-1H-pyrazole-4-carboxamide methanesulfonate, as obtained in Example 1 (form 1 of the mesylate salt of compound (X)).

FIG. 2: Powder XRD diffractogram of form 2 of the mesylate salt of compound (X).

FIG. 3: Powder XRD diffractogram of form 3 of the mesylate salt of compound (X).

FIG. 4: Powder XRD diffractogram of crystalline compound (IX).

FIG. 5: Powder XRD diffractogram of crystalline tent-butyl 4-(3-carbamoyl-2-(4-phenoxyphenyl)pyrazolo[1,5-a]pyrimidin-7-yl)piperidine-1-carboxylate (compound VII wherein PG is tert-butyloxycarbonyl); as obtained in Example 3C.

### DETAILED DESCRIPTION OF THE INVENTION

The present disclosure provides new procedures and intermediates for the preparation of Zanubrutinib or any salt thereof.

As discussed above, the processes described in the literature have a number of disadvantages. For example, the use of column/chiral chromatography techniques for the purification and separation of intermediates. Also, the ester group is converted to amide group only in the later stages of the preparation of Zanubrutinib, which result in a yield loss in the final stage. By way of contrast, the processes of the present disclosure do not convert the ester group to amide group in the last stages, thus avoiding the yield loss in this conversion at a late stage, thereby making the process more economical for application on an industrial scale.

As used herein, and unless indicated otherwise, the term “isolated” in reference to the intermediates of the present disclosure, their salts or solid state forms thereof corresponds to compounds that are physically separated from the reaction mixture in which they are formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

As used herein in any of the disclosed processes, and unless indicated otherwise, the term “one pot process” refers to a continuous process for preparing a desired product, in which penultimate product is converted to the desired product in the same vessel.

As used herein, in any of the disclosed processes or process steps, unless indicated otherwise, the term “anhydrous” refers to non-aqueous conditions, particularly wherein the reaction mixture contains less than 2 wt % water, less than 1.5 wt % water, less than 1 wt % water, less than 0.5 wt % water, less than 0.25 wt % water, less than 0.15 wt % water, less than 0.1 wt % water, or less than 0.05 wt % water.

The processes or steps may be referred to herein as being carried out “overnight.” This refers to time intervals, e.g., for the processes or steps, that span the time during the night, when the processes or steps may not be actively observed. The time intervals are from about 8 to about 20 hours, or about 10 to about 18 hours, or about 16 hours.

As used herein, in any of the disclosed processes, and unless indicated otherwise, the term “reduced pressure” refers to a pressure of about 10 mbar to about 500 mbar, or about 50 mbar.

The amount of solvent employed in chemical processes, e.g., reactions or crystallizations, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding MTBE (1.5 v/v) to a 100 ml reaction mixture would indicate that 150 mL of MTBE was added.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of compound referred to herein as being characterized by graphical data “as depicted in” a Figure will thus be understood to include any crystal forms of the compound, characterized with the graphical data having such small variations, as are well known to the skilled person, in comparison with the Figure.

As used herein, unless stated otherwise, XRPD pattern peaks reported herein are optionally measured using CuK_{α} radiation, λ=1.5418 Å. Preferably, XRPD peaks reported herein are measured using CuK_{α} radiation, λ=1.54 Å, at a temperature of 25±3° C.

As used herein, in any of the disclosed processes or compounds, and unless indicated otherwise, the term “substantially pure” relates to a compound, having a purity, measured as % area HPLC, of about 95% or more. In some embodiments, the term relates to compounds having a purity of about 95% or more. In other embodiments, the term relates to compounds having a purity of about 97% area by HPLC. In further embodiments, the term relates to compounds having a purity of about 99% area by HPLC. In yet other embodiments, the term relates to compounds having a purity of about 99.3% area by HPLC. In still further embodiments, the term relates to compounds having a purity of about 99.8% area by HPLC.

As used herein, in any of the disclosed processes or compounds, and unless indicated otherwise, the term “substantially free of”, referring to a compound of the present disclosure such as compounds I or II, or to any of the solid state forms of the present disclosure, means that the compounds or the solid state form contain about 20% (w/w) or less, about 10% (w/w) or less, about 5% (w/w) or less, about 4% (w/w) or less, about 3% (w/w) or less, about 2% (w/w) or less, about 1% (w/w) or less, about 0.5% (w/w) or less, or about 0.2% (w/w) or less or 0% of a specified impurity or any other forms of the subject compound as measured, for example, by XRPD, respectively. Thus, compounds or solid state form of compounds described herein as is substantially free of a specified impurity or any other solid state forms respectively, would be understood to contain greater than about 80% (w/w), greater than about 90% (w/w), greater than about 95% (w/w), greater than about 98% (w/w), greater than about 99% (w/w), or 100% of the subject compound or solid state form of compound. Accordingly, in some embodiments of the disclosure, the described compounds and solid state forms of intermediates may contain from about 1% to about 20% (w/w), from about 5% to about 20% (w/w), or from about 5% to about 10% (w/w) of one or more specified impurity or other solid state forms of the same intermediate, respectively.

As used herein, in any of the disclosed processes, and unless indicated otherwise, the term “polar aprotic solvent” has a dielectric constant greater than 15 and is at least one selected from the group consisting of amide-based organic solvents, such as N,N-dimethylformamide (DMF), N,N-dimethylacetamide (DMA or DMAc), N-methylpyrrolidone (NMP), formamide, acetamide, propanamide, hexamethyl phosphoramide (HMPA), and hexamethyl phosphorus triamide (HMPT); nitro-based organic solvents, such as nitromethane, nitroethane, nitropropane, and nitrobenzene; pyridine-based organic solvents, such as pyridine and picoline; sulfone-based solvents, such as dimethylsulfone, diethylsulfone, diisopropylsulfone, 2-methylsulfolane, 3-methylsulfolane, 2,4-dimethylsulfolane, 3,4-dimethy sulfolane, 3-sulfolene, and sulfolane; and sulfoxide-based solvents such as dimethylsulfoxide (DMSO).

As used herein, in any of the disclosed processes, and unless indicated otherwise, the term “ether solvent” is an organic solvent containing an oxygen atom —O— bonded to two other carbon atoms. “Ether solvents” include, but are not limited to, diethyl ether, diisopropyl ether, methyl t-butyl ether (MTBE), glyme, diglyme, tetrahydrofuran (THF), 2-methyltetrahydrofuran (2-Me THF), 1,4-dioxane, dibutyl ether, dimethylfuran, 2-methoxyethanol, 2-ethoxyethanol, anisole, C₂-C₆ ethers, or the like.

As used herein, in any of the disclosed processes, and unless indicated otherwise, the term “ester solvent” is an organic solvent containing a carboxyl group —(C═O)—O— bonded to two other carbon atoms. “Ester solvents” include, but are not limited to, ethyl acetate, n-propyl acetate, isopropyl acetate, n-butyl acetate, isobutyl acetate, t-butyl acetate, ethyl formate, methyl acetate, methyl propanoate, ethyl propanoate, methyl butanoate, ethyl butanoate, C₃-C₆ esters, or the like.

As used herein, in any of the disclosed processes, and unless indicated otherwise, the term “ketone solvent” is an organic solvent containing a carbonyl group —(C═O)— bonded to two other carbon atoms. “Ketone solvents” include, but are not limited to, acetone, ethyl methyl ketone, diethyl ketone, methyl isobutyl ketone (MIBK), C₃-C₆ ketones, or the like.

As used herein, in any of the disclosed processes, and unless indicated otherwise, the term “nitrile solvent” is an organic solvent containing a cyano —(C≡N) bonded to another carbon atom. “Nitrile solvents” include, but are not limited to, acetonitrile, propionitrile, C₂-C₆ nitriles, or the like.

As used herein, in any of the disclosed processes, and unless indicated otherwise, the term “alcohol” as applied to solvents include a straight, branched or cyclic hydrocarbon containing at least one hydroxyl group, and may also include other substituents such as halo (particularly fluoro or chloro), alkoxy, or nitro. Typical alcohol solvents include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol i.e. IPA), 2-methoxyethanol, 1-butanol, 2-butanol, iso-butyl alcohol, tert-butyl alcohol, 2-ethoxyethanol, diethylene glycol, 1-, 2-, or 3-pentanol, neo-pentyl alcohol, tert-pentyl alcohol, diethylene glycol monomethyl ether, diethylene glycol monoethyl ether, cyclohexanol, and glycerol.

As used herein, in any of the disclosed processes, and unless indicated otherwise, the term “hydrocarbon solvent” refers to a liquid, saturated hydrocarbon, which may be linear, branched, or cyclic. It is capable of dissolving a solute to form a uniformly dispersed solution. Examples of a hydrocarbon solvent include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, C₅-C₈ aliphatic hydrocarbons, and mixtures thereof.

As used herein, in any of the disclosed processes, and unless indicated otherwise, the term “aromatic hydrocarbon” as applied to a solvent, refers to a liquid, unsaturated, cyclic, hydrocarbon containing one or more rings which has at least one 6-carbon ring containing three double bonds. Examples of an aromatic hydrocarbon solvent include, but are not limited to, benzene, toluene, ethylbenzene, m-xylene, o- xylene, p-xylene, indane, naphthalene, tetralin, and trimethylbenzene (e.g. mesitylene).

As used herein, in any of the disclosed processes, and unless indicated otherwise, the term “halogenated hydrocarbon” as applied to a solvent, refers to a liquid, saturated or unsaturated, cyclic or straight chain, hydrocarbon solvent, which may comprise an alkane, aromatic hydrocarbon or cyclic alkane, which is substituted with at least one halo group, particularly fluoro or chloro. Suitable halogenated hydrocarbon solvents include: a C₁-C₆ chlorinated hydrocarbons, such as dichloromethane and dichloroethane, carbon tetrachloride, chlorobenzene, fluorobenzene, trifluorotoluene, chloroform, dichloromethane, and dichloroethane.

As used herein, in any of the disclosed processes, and unless indicated otherwise, the term “aromatic alcohol” as applied to a solvent, refers to a liquid, unsaturated, cyclic, hydrocarbon containing one or more rings which has at least one 6-carbon ring containing three double bonds, and at least one hydroxyl group. An example of an aromatic alcohol solvent is benzyl alcohol.

As used herein in any of the disclosed processes or compounds, unless otherwise indicated, “alkyl” refers to a monoradical of a branched or unbranched saturated hydrocarbon chain and can be substituted or unsubstituted. Particularly, alkyl groups may contain 1-6 carbon atoms or optionally 1-4 carbon atoms. In Examples of alkyl groups include, but are not limited to, methyl, ethyl, n-propyl, n-butyl, n-pentyl, n-hexyl, n-heptyl, n-octyl, n-nonyl, n-decyl, isopropyl, tert-butyl, isobutyl, etc.

As used herein, unless otherwise indicated, in any of the disclosed processes or compounds, “alkoxy” refers to the O-(alkyl) group where the alkyl group is defined above.

As used herein, in any of the disclosed processes, and unless indicated otherwise, the term “organic base” is an organic compound, which acts as a base. Examples of such bases include, but are not limited to, trimethylamine (TEA), pyridine, diisopropylamine (DIPA), N,N-diisopropylethylamine (DIPEA or DIEA), N-methylmorpholine (NMM), 1,4-diazabicyclo[2.2.2]octane (DABCO), triethanolamine, tributylamine, lutidine, 4-dimethylamino pyridine (DMAP), diethanolamine, 4-methylmorpholine, dimethylethanolamine, tetra methylguanidine, morpholine, imidazole, 2-methylimidazole, 4-methylimidazole, tetra methylammonium hydroxide, tetraethylammonium hydroxide, N-methyl-1,5,9-triazabicyclo[4.4.0]decene, 1,8-diazabicyclo[5.4.0]undec-7-ene (DBU), dicyclo hexylamine, and picoline.

As used herein, in any of the disclosed processes or compounds, and unless indicated otherwise, the term “protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996).

A process according to one aspect of the disclosure is set out in Scheme C below:

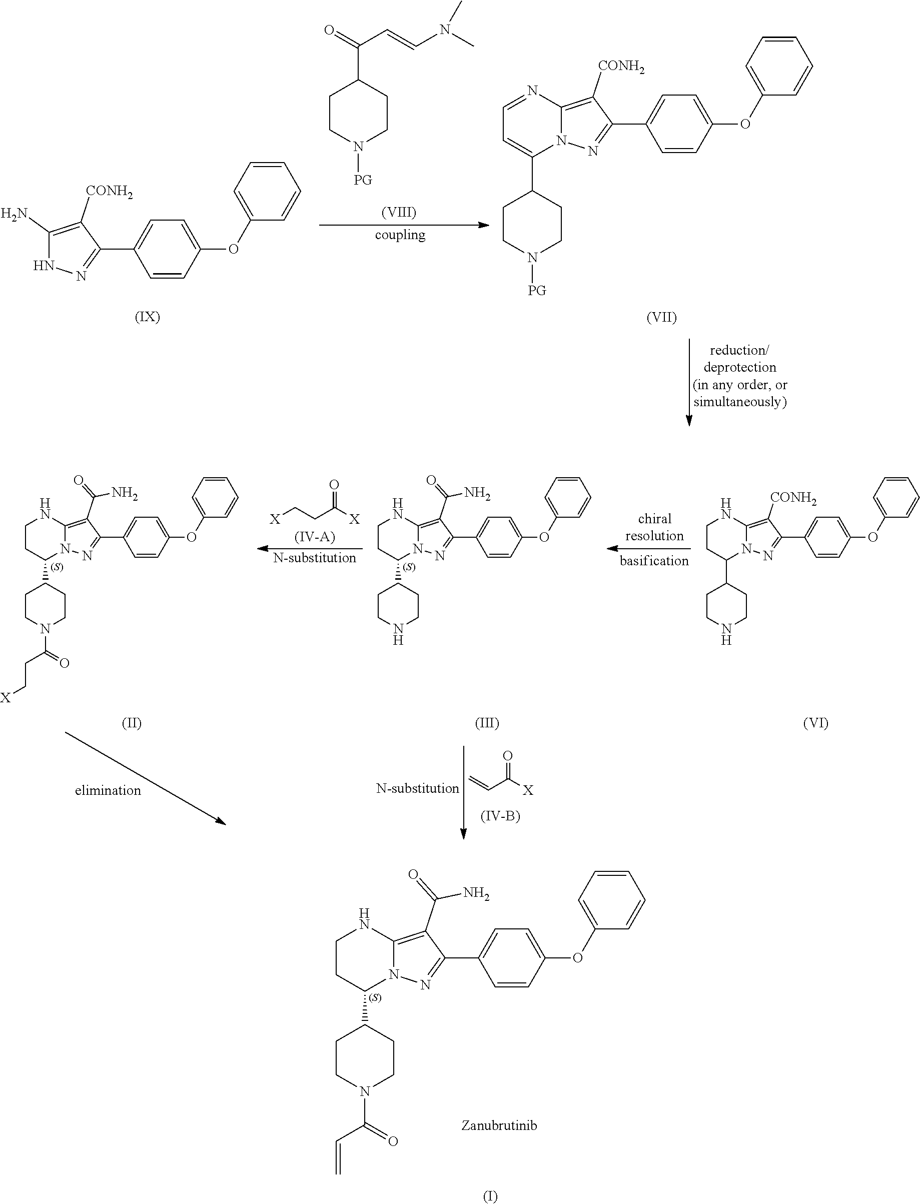

wherein PG is a protecting group; and X is chloro or bromo [wherein X in formula (IV-A) can be the same or different]. The steps of coupling, reduction, deprotection, chiral resolution, N-substitution and elimination in the above process, can be carried out according to any of the methods disclosed herein.

It can be seen that a key feature is the use of amide-substituted compounds/intermediates, thereby avoiding the need to carry out this conversion at the end of the synthesis. The starting material (IX) may be prepared by hydrolysis of the corresponding cyano-substituted compound of formula (XI):

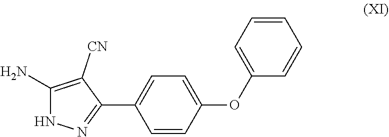 [SMILES: N#Cc1c(-c2ccc(Oc3ccccc3)cc2)n[nH]c1N]

The hydrolysis step is described below.

Chiral resolution of the compound of formula (VI) may be carried out by any suitable process, such as diastereomeric salt separation, i.e. via formation of a salt with an optically active acid, thereby forming a compound of formula (V):

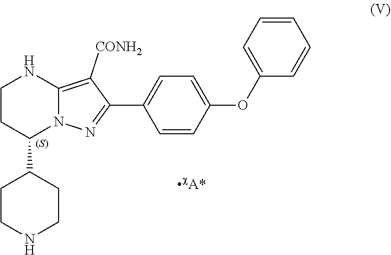

wherein A* is a chiral acid and x is 0.5 for a dibasic acid, and 1 for a monobasic acid or malic acid. For example, the resulting chiral salts may be the L-DBTA salt, the L-DPTT salt and the L-malate salt:

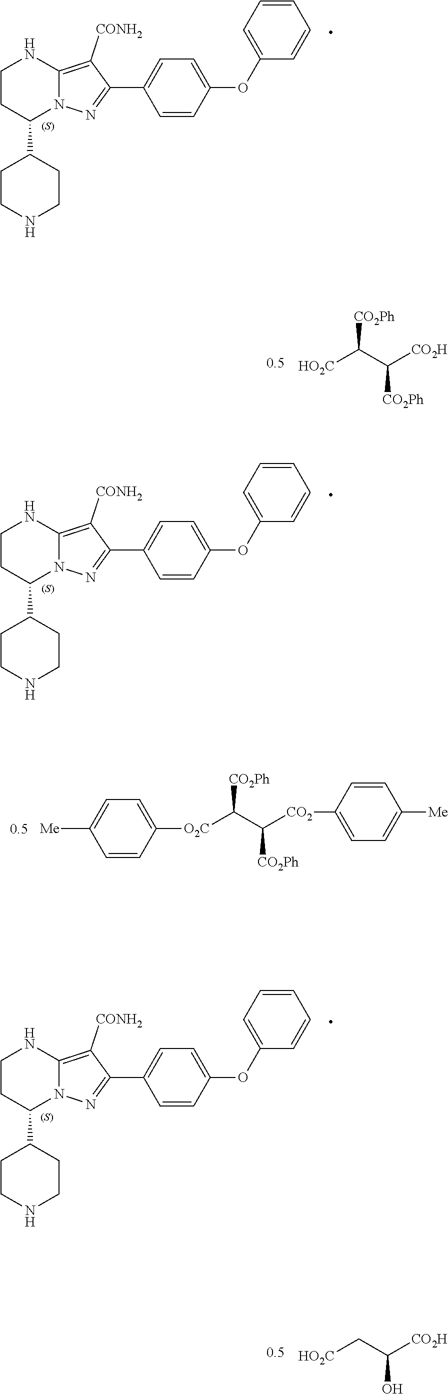

The present disclosure therefore additionally relates to any of the above compounds, i.e. chiral salts of formula (V). Any of these chiral salts are useful as intermediates for the preparation of Zanubrutinib or salts thereof. The chiral salt may be basified to form the compound (III):

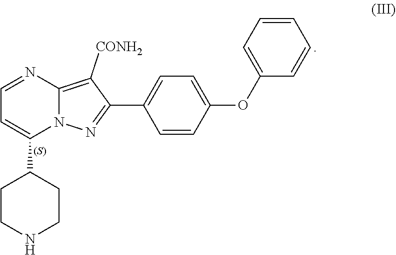

Compound (III) may be isolated and used in the next step (N-substitution). Alternatively, the chiral salt (V) may be basified to produce a reaction mixture comprising Compound (III). The reaction mixture comprising Compound (III) may be used in the next step (N-substitution) in situ, i.e. without isolating Compound (III).

In the above process, the conversion of compound (VII) to (VI) may be carried out by sequential reduction followed by deprotection, sequential deprotection followed by reduction, or by a simultaneous reduction/deprotection, i.e.:

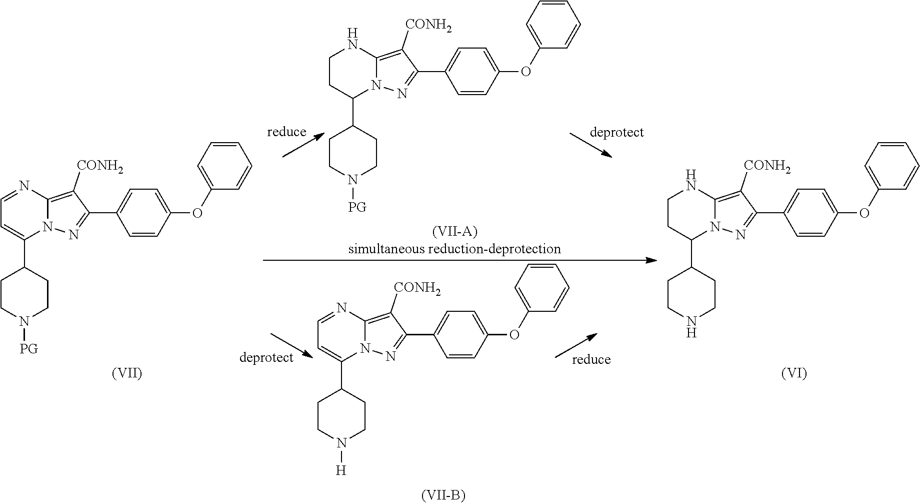

Optionally, the conversion of compound (VII) to (VI) may be carried out by reducing the compound of formula (VII) to form a compound of formula (VII-A), and deprotecting the compound of formula (VII-A) to produce a compound of formula (VI). The sequential steps of reduction and deprotection or deprotection and reduction may be carried out as a one-pot reaction, i.e. without isolation of the compounds (VII-A) or (VII-B) as solids

Another aspect of the present disclosure provides a process for preparing Zanubrutinib or a salt thereof, which comprises a step of reacting a compound (IX) with compound (VIII) to obtain compound (VII):

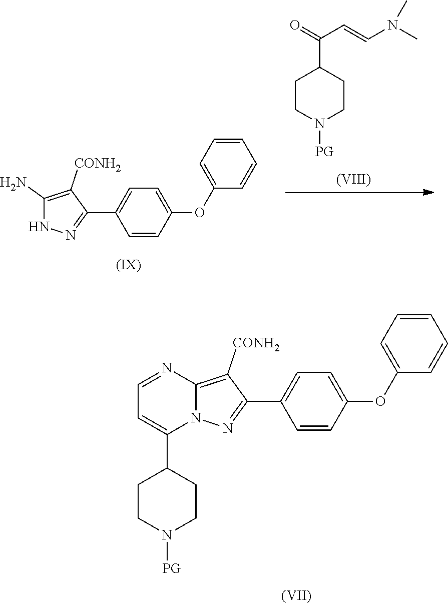

wherein PG is a protecting group; and converting the compound of formula (VII) to Zanubrutinib or a salt thereof.

In any of the processes or compounds described herein, the protecting group PG may be selected from acetyl, benzyl, methyl, benzoyl, toluoyl, methoxycarbonyl, ethoxycarbonyl, benzyloxycarbonyl, tert-butyloxycarbonyl, allyloxycarbonyl, 4-methoxybenzyl, para-methoxybenzylcarbonyl, 3,4-dimethoxybenzyoyl, propionyl, butyryl, phenylacetyl, phenoxyacetyl, trityl, 2,2,2-trichloroethoxycarbonyl, carbobenzoxy, 4-methoxybenzyloxycarbonyl, 9-fluorenylmethoxycarbonyl, 2-iodoethoxycarbonyl, 4-methoxy-2,3,6-trimethylbenzenesulfonyl, methanesulfonyl, para-toluenesulfonyl, phenyl sulfonyl, trifluorocarbonyl, and 2-trimethylsilylethoxycarbonyl, 4-nitrobenzenesulfonyl. In embodiments of the disclosure, PG may be optionally selected from: methoxycarbonyl, ethoxycarbonyl, benzyloxycarbonyl, tert-butyloxycarbonyl, para-methoxybenzylcarbonyl, 3,4-dimethoxybenzyoyl, phenylacetyl, phenoxyacetyl, or 4-methoxybenzyloxycarbonyl. Particularly, in any of the processes or compounds described herein, PG is tert-butyloxycarbonyl.

The reaction of the compound of formula (IX) with (VIII) to form the compound of formula (VII) according to any of the herein described processes, may be carried out in the presence of an acid, optionally an organic acid, wherein the acid is optionally selected from the group consisting of acetic acid or trifluoroacetic acid. Optionally the reaction is carried out in the present of acetic acid, particularly glacial acetic acid. The coupling reaction according to any aspect or embodiment of the disclosure may optionally be carried out under anhydrous, i.e. non-aqueous, conditions.

The reaction of the compound of formula (IX) with (VIII) to form the compound of formula (VII) according to any of the herein described processes, may be typically carried out in any suitable solvent, such as a polar aprotic solvent or a polar protic solvent, optionally a polar aprotic solvent, although any suitable solvent can be used. Suitable solvents for this conversion maybe selected from: an aromatic hydrocarbon, a halogenated hydrocarbon, an aromatic alcohol, a ketone, an ester, an ether, an alcohol, a nitrile, or a polar aprotic solvent such as N,N-dimethylformamide, N-methylpyrrolidone, or DMSO. Particularly, suitable solvents may be selected from the group consisting of: toluene, mesitylene, chlorobenzene, benzonitrile, benzyl alcohol, xylene, N,N-dimethylformamide (DMF), acetonitrile (ACN), methylethylketone (MEK), tetrahydrofuran (THF), dimethylacetate (DMAc), dimethylsulfoxide (DMSO), N-methylpyrrolidone (NMP), acetone, methylisobutylketone (MIBK), dichloromethane (DCM), methanol, ethanol, 1-propanol, 2-propanol and 1-butanol.

The reaction of the compound of formula (IX) with (VIII) to form the compound of formula (VII) according to any of the herein described processes, may optionally be carried out using glacial acetic acid and an alcohol solvent, particularly propanol or butanol. Optionally, the reaction of compound of formula (IX) with (VIII) to form the compound of formula (VII) according to any of the herein described processes, may be carried out using trifluoroacetic acid and a nitrile solvent, particularly acetonitrile solvent.

Alternatively, the reaction of the compound of formula (IX) with (VIII) to form the compound of formula (VII) according to any of the herein described processes, may be carried out using trifluoroacetic acid and an aprotic solvent, such as toluene, mesitylene, chlorobenzene, benzonitrile, benzyl alcohol, xylene, N,N-dimethylformamide (DMF), acetonitrile (ACN), methylethylketone (MEK), tetrahydrofuran (THF), dimethylacetate (DMAc), dimethylsulfoxide (DMSO), N-methylpyrrolidone (NMP), acetone, methylisobutylketone (MIBK), or dichloromethane.

The reaction of the compound of formula (IX) with (VIII) to form the compound of formula (VII) according to any of the herein described processes, particularly wherein the acid is acetic acid, may be typically carried out with heating, particularly to a temperature of: about 50° C. to about 130° C., about 70° C. to about 120° C., about 80° C. to about 110° C., about 90° C. to about 100° C., or about 95° C. The reaction mixture may be heated for: about 6 to 24 hours, about 8 to about 20 hours, or about 10 to about 14 hours, or about 12 hours.

Alternatively, the reaction of the compound of formula (IX) with (VIII) to form the compound of formula (VII) according to any of the herein described processes, particularly wherein the acid is trifluoroacetic acid, may be typically carried out in slightly milder conditions, for example by heating, particularly to a temperature of: about 25° C. to about 60° C., about 30° C. to about 50° C., about 35° C. to about 45° C., or about 40° C. The reaction mixture may be heated for: about 0.5 to 10 hours, about 1 to about 5 hours, or about 2 to about 4 hours, or about 3 hours.

The reaction mixture may be subsequently cooled to, particularly to a temperature in the range of: about 10° C. to about 40° C., about 15° C. to about 30° C. or about 20° C. to about 25° C., and optionally maintained for about 5 to about 20 hours, about 8 to about 15 hours, or about 10 hours.

Advantageously, the compound (VII) can be isolated in high purity directly from the reaction mixture. For example, following the cooling step described in the preceding paragraph, the mixture may be further cooled to: −10° C. to 10° C., −5° C. to 8° C., or about 0° C. to 5° C. Typically, the cooling may result in the precipitation of crystals of compound (VII) having high purity to be directly isolated from the cooled mixture. The resulting compound of formula (VII) may be isolated in high yield by filtration. Due to its high purity, the compound (VII) can be used directly in subsequent steps, without any further purification.

In any aspect of the present disclosure, there is provided a crystalline form of compound (VII):

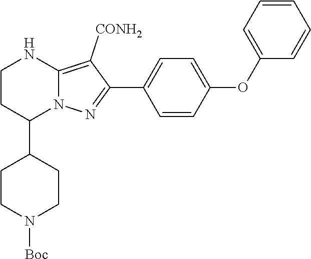 [SMILES: CC(C)(C)OC(=O)N1CCC(C2CCNc3c(C(N)=O)c(-c4ccc(Oc5ccccc5)cc4)nn32)CC1]

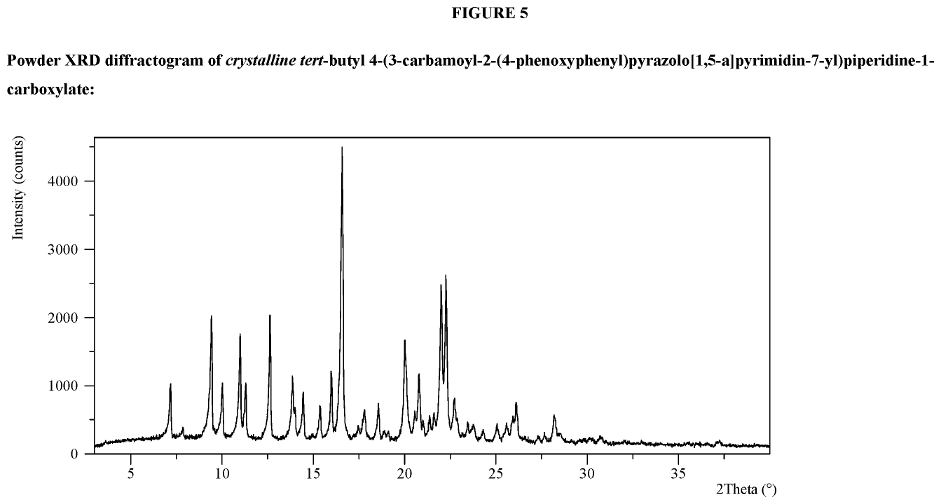

which may be characterized by data selected from one or more of the following: an X-ray powder diffraction pattern having peaks at 7.2, 9.4, 10.0, 12.6 and 16.6 degrees 2-theta±0.2 degrees 2-theta; an X-ray powder diffraction pattern substantially as depicted in FIG. 5; and combinations of these data. The crystalline form of compound (VII) can be further characterized by an X-ray powder diffraction pattern having peaks at 7.2, 9.4, 10.0, 12.6 and 16.6 degrees 2-theta±0.2 degrees 2-theta and also having any one, two, three, four or five additional peaks selected from 11.0, 11.3, 14.5, 16.0 and 20.0 degrees 2-theta±0.2 degrees 2-theta.

As discussed above, according to any of the processes disclosed herein, the compound of formula (VII) can be converted to a compound of formula (VI) by reduction and deprotection steps, which may be carried out sequentially in any order or may be carried out simultaneously:

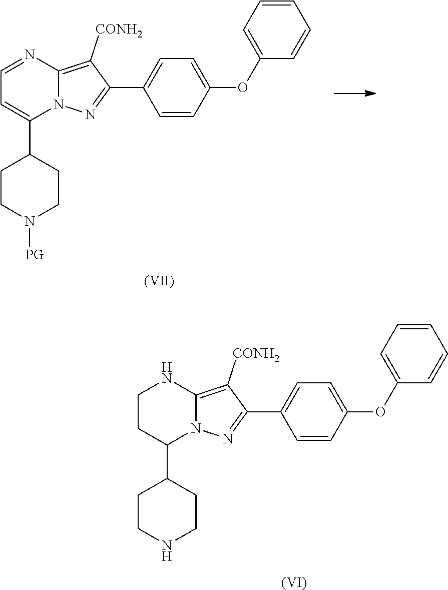

According to one option, the compound of formula (VII) is reduced to form a compound of formula (VII-A):

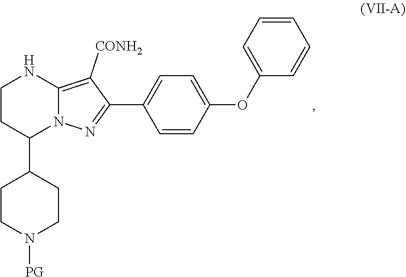

and the compound (VII-A) is deprotected to form a compound of formula (VI), optionally wherein the deprotection is carried out without isolation of the compound of formula (VII-A).

According to another option, the compound of formula (VII) is deprotected to form a compound (VII-B):

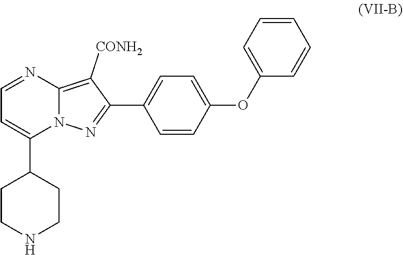 [SMILES: NC(=O)c1c(-c2ccc(Oc3ccccc3)cc2)nn2c(C3CCNCC3)ccnc12]

and reducing the compound (VII-B) to form the compound (VI), optionally wherein the reduction is carried out without isolation of the compound of formula (VII-B).

According to yet another option, the compound of formula (VII) is reduced to form a compound of formula (VII-A) by simultaneously reducing and deprotecting the compound (VII) to form the compound (VI).

The reduction step in the conversion of the compound of formula (VII) to (VI), whether it is carried out before or after the deprotection step, or simultaneously with the deprotection step, may be carried out by hydrogenation in the presence of hydrogen gas and a catalyst, optionally where the catalyst comprises palladium or platinum, or wherein the reduction is carried out with a reducing agent, optionally sodium borohydride or sodium cyanoborohydride.

The hydrogenation reaction may be carried out in the presence of an acid, optionally an organic acid, optionally acetic acid (e.g. glacial acetic acid); or optionally wherein the acid is malic acid, mandelic acid, camphorsulfonic acid, tartaric acid, dibenzoyltartaric acid, di-p-toluoyltartaric acid, or optically active isomers thereof; optionally wherein the acid is L-malic acid, L-mandelic acid, L-camphorsulfonic acid, L-tartaric acid, dibenzoyl-L-tartaric acid, di-p-toluoyl-L-tartaric acid, D-malic acid, D-mandelic acid, D-camphorsulfonic acid, D-tartaric acid, dibenzoyl-D-tartaric acid, di-p-toluoyl-D-tartaric acid.

The hydrogenation reaction may be carried out in anhydrous, i.e. non-aqueous conditions, particularly wherein the reaction mixture contains less than 2 wt % water, less than 1.5 wt % water, less than 1 wt % water, less than 0.5 wt % water, less than 0.25 wt % water, less than 0.15 wt % water, less than 0.1 wt % water, or less than 0.05 wt % water.

The hydrogenation reaction as described herein may be carried out in any suitable solvent, particularly aprotic solvents. Suitable solvents include those selected from THF, DMSO, or dioxane, or mixtures thereof, and particularly THF.

The hydrogenation reaction as described herein may advantageously be carried under mild conditions, for example, at a pressure of: about 1 bar to about 10 bar, about 2 bar to about 9 bar, about 3 bar to about 8 bar, about 4 bar to about 7 bar, about 5 to about 6 bar. Optionally, the hydrogenation is carried out at a temperature of about 20° C. to about 100° C., about 30° C. to about 90° C., about 40° C. to about 80° C., about 50° C. to about 70° C., or about 60° C.

The hydrogenation reaction as described herein may be carried out for a period of: about 10 hours to about 48 hours, about 14 hours to about 40 hours, about 18 hours to about 36 hours, about 20 hours to about 30 hours, or about 24 hours.

Advantageously, the product from the hydrogenation reaction can be isolated directly from the reaction mixture, typically as an acid addition salt due to the presence of an acid when used. The acid addition salt may precipitate directly from the hydrogenation reaction mixture, and can be readily isolated by e.g. filtration.

The deprotection step comprises removing the PG protecting group, optionally followed by neutralization if the deprotection is carried out using acid. The deprotection step may be carried out using any reagent suitable for removing the PG group as well known to the skilled person. For example, an acid cleavable PG group may be cleaved by a mineral acid. Optionally, for example, when PG is Boc, a mineral acid, such as hydrochloric acid, may be used. The use of an acid may give rise to the product being formed as an acid addition salt.

The deprotection step in the conversion of the compound of formula (VII) to (VI), whether it is carried out before or after the reduction step, or simultaneously with the reduction step, may be carried out using an acid (e.g. a mineral acid, such as hydrochloric acid) or a base. Any suitable polar solvent may be used for the deprotection reaction. Optionally the solvent is an alcohol, for example propanol, such as 2-propanol. Typically the solvent may be used in an amount of: 10 to 40 volumes, 15 to 30 volumes or about 20 volumes relative to the starting compound. The deprotection reaction may be carried out by heating a solution of the starting material in the solvent and deprotection agent, such as an acid, to a temperature of: 30° C. to 100° C., 40° C. to 80° C., 50° C. to 70° C., or about 60° C. The reaction mixture may be maintained for: 0.5 to 4 hours, 0.8 to 3 hours, 1 to 2 hours, or about 1.5 hours.

If required, the basification (neutralization) step following deprotection with an acid, may advantageously carried out in one pot, for example by basification of the deprotection reaction mixture, optionally using an alkali metal hydroxide, optionally potassium hydroxide, or sodium hydroxide, or an alkali metal carbonate, optionally sodium carbonate, potassium carbonate, or an alkali metal hydrogen carbonate, optionally sodium hydrogen carbonate or potassium hydrogen carbonate. Typically sodium hydroxide may be used. The amount of base may be added in a sufficient quantity to adjust the pH of the reaction mixture to: 11 to 14, 11.5 to 13, 12 to 12.8, about 12.6±0.2 or about 12.6±0.1

The deprotected (optionally following neutralization as required) product may be isolated from the reaction mixture by extraction (e.g. into dichloromethane) and subsequent precipitation from an antisolvent (e.g. n-heptane). Advantageously, the deprotected product can be obtained in high yield, and high purity, thus providing a product which is highly suitable for further process steps without the need to conduct further purification procedures.

The amount of base may be added in a sufficient quantity to adjust the pH of the reaction mixture to around neutral, i.e. 7±0.5, 7±0.2, 7±0.1, or about 7. The reaction may be carried out at about room temperature. The mixture is preferably stirred at about room temperature for: 0.2 to 4 hours, 0.4 to 2 hours, or 0.5 to 1.5 hours, or about 1 hour. Advantageously, the product compound (X) typically precipitates as crystals having high purity, directly from the reaction mixture. The compound (X) may be used in the subsequent steps without further purification.

Alternatively, depending on the protecting group PG, deprotection may be effected simultaneously with the reduction step. For example, a protecting group PG that is cleavable under the hydrogenation conditions in embodiments of the reduction step may be used.

The starting material for the above-described process of the present disclosure, i.e. the compound of formula (IX), may be advantageously obtained by basification of an acid addition salt of a compound of formula (X):

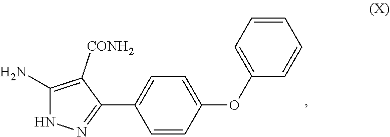 [SMILES: NC(=O)c1c(-c2ccc(Oc3ccccc3)cc2)n[nH]c1N]

wherein the salt is with methanesulfonic acid, p-toluene sulfonic acid or polyphosphoric acid. The preparation and hydrolysis of the acid addition salt of the compound of formula (X) is described below and advantageously facilitates purification of the compound (IX). Particularly, the acid addition salt of the compound of formula (X) is prepared under anhydrous conditions. The basification (neutralization) reaction may be carried out using an inorganic base, such as an alkali metal hydroxide, optionally potassium hydroxide, or sodium hydroxide, or an alkali metal carbonate, optionally sodium carbonate, potassium carbonate, or an alkali metal hydrogen carbonate, optionally sodium hydrogen carbonate or potassium hydrogen carbonate. The basification (neutralization) reaction is advantageously carried out in water, enabling the resulting compound of formula (IX) to be isolated as a crystalline product directly from the reaction mixture. The reaction may be carried out in water, optionally: 2 to 20 volumes, 5 to 15 volumes, 8 to 12 volumes, or about 10 volumes. The amount of base may be added in a sufficient quantity to adjust the pH of the reaction mixture to around neutral, i.e. 7±0.5, 7±0.2, 7±0.1, or about 7. The reaction may be carried out at about room temperature. The mixture is preferably stirred at about room temperature for: 0.2 to 4 hours, 0.4 to 2 hours, or 0.5 to 1.5 hours, or about 1 hour. Advantageously, the product compound (X) typically precipitates as crystals having high purity, directly from the reaction mixture. The compound (X) may be used in the subsequent steps without further purification.

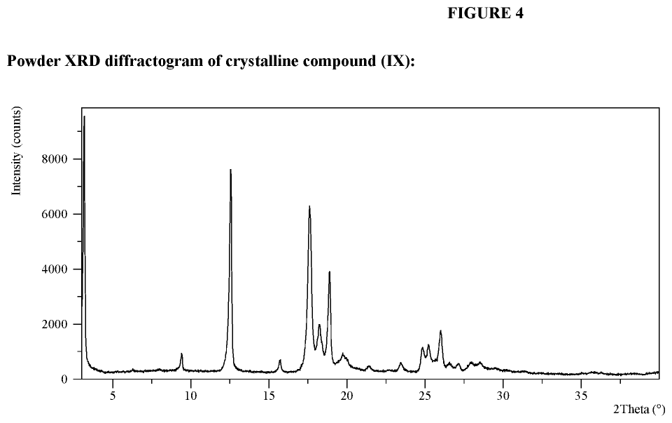

In any aspect of the present disclosure, there is provided a crystalline form of compound (IX). The crystalline form of compound (IX) may be characterized by an X-ray powder diffraction pattern having peaks at 3.2, 9.4, 12.6, 17.6 and 18.9 degrees 2-theta±0.2 degrees 2-theta. The crystalline form of compound (IX) may be optionally or additionally characterized by an X-ray powder diffraction pattern substantially as depicted in FIG. 4. The crystalline form of compound (IX) can be further characterized by an X-ray powder diffraction pattern having peaks at 3.2, 9.4, 12.6, 17.6 and 18.9 degrees 2-theta±0.2 degrees 2-theta, and also having any one, two, three, four or five additional peaks selected from 15.7, 18.2, 24.8, 25.2 and 26.0 degrees 2-theta±0.2 degrees 2-theta. Alternatively the crystalline form of compound (IX) may be characterized by an X-ray powder diffraction pattern having peaks at: 3.2, 9.4, 12.6, 15.7, 17.6, 18.2, 18.9, 24.8, 25.2, and 26.0 degrees 2-theta±0.2 degrees 2-theta. The crystalline form of compound (IX) may be obtained by any of the processes described herein.

In one aspect of the disclosure, the compound of formula (X) may be prepared by hydrolysis of a compound of formula (XI):

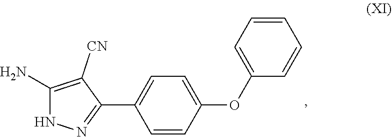 [SMILES: N#Cc1c(-c2ccc(Oc3ccccc3)cc2)n[nH]c1N]

optionally by acid hydrolysis, and particularly by acid hydrolysis under anhydrous conditions, followed by basification. In any aspect or embodiment of the disclosure, the hydrolysis may be carried out in anhydrous conditions using a neat liquid organic acid, preferably neat methanesulfonic acid. The resulting product is the acid addition salt of the compound (X). The hydrolysis reaction may be carried out by forming a solution of the compound (XI) in a neat liquid organic acid, particularly methanesulfonic acid and heating. The amount of acid may typically be: from 2 to 10 volumes, from 3 to 8 volumes, from 4 to 6 volumes, and optionally about 5 volumes relative to the starting material compound (XI). The mixture of compound (X) and the acid may be heated to a temperature of: 30° C. to 100° C., 40° C. to 80° C., 50° C. to 70° C., or about 60° C. The reaction mixture may be maintained at this temperature range for: 4 to 28 hours, 10 to 24 hours, 15 to 20 hours, or about 18 hours. After cooling, typically to about room temperature, the reaction mixture may be diluted with water, which advantageously allows the resulting acid salt of compound (X) to crystallise or precipitate directly from the reaction mixture. For example, the hydrolysis using methanesulfonic acid results in the production of a methanesulfonate salt of the compound (X),

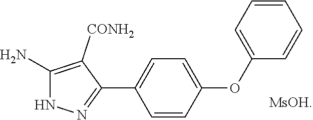 [SMILES: NC(=O)c1c(-c2ccc(Oc3ccccc3)cc2)n[nH]c1N]

Advantageously, this compound can readily be isolated directly from the reaction mixture as a crystalline solid having high purity, which significantly reduces the carry over of impurities in the synthesis, thereby resulting in improved purities and yields in the subsequent steps in the process for preparing Zanubrutinib. The basification step may be carried out as described above.

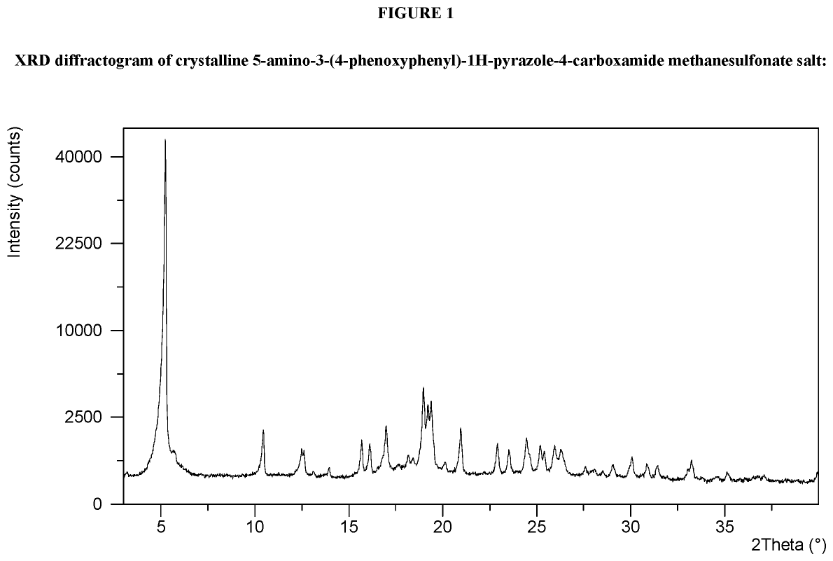

In any aspect of the present disclosure, there is provided a crystalline form of the mesylate salt of compound (X); designated form 1. The crystalline form of the mesylate salt of compound (X) may be characterized by an X-ray powder diffraction pattern having peaks at 5.2, 10.5, 17.0, 19.0, and 21.0 degrees 2-theta±0.2 degrees 2-theta. The crystalline form 1 of the mesylate salt of compound (X) may be optionally or additionally characterized by an X-ray powder diffraction pattern substantially as depicted in FIG. 1. Crystalline form 1 of compound (X) can be further characterized by an X-ray powder diffraction pattern having peaks at 5.2, 10.5, 17.0, 19.0, and 21.0 degrees 2-theta±0.2 degrees 2-theta, and also having any one, two, three, four or five additional peaks selected from 15.7, 16.1, 19.4, 22.9 and 23.5 degrees 2-theta±0.2 degrees 2-theta. Alternatively, crystalline form I of compound (X) may be characterized by an X-ray powder diffraction pattern having peaks at 5.2, 10.5, 15.7, 16.1, 17.0, 19.0, 19.4, 21.0, 22.9, and 23.5 degrees 2-theta±0.2 degrees 2-theta.

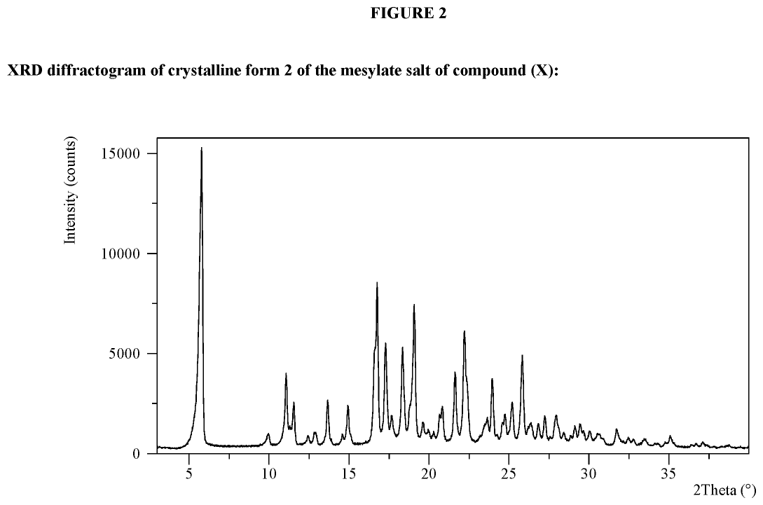

In a further embodiment the invention provides a crystalline form of the mesylate salt of compound (X); designated form 2. The crystalline form 2 can be characterized by an X-ray powder diffraction pattern having peaks at 5.8, 11.1, 13.7, 17.3 and 18.3 degrees 2-theta ±0.2 degrees 2-theta. The crystalline form 2 of the mesylate salt of compound (X) may be optionally or additionally characterized by an X-ray powder diffraction pattern substantially as depicted in FIG. 2. Crystalline form 2 can be further characterized by an X-ray powder diffraction pattern having peaks at 5.8, 11.1, 13.7, 17.3 and 18.3 degrees 2-theta+0.2 degrees 2-theta, and also having any one, two, three, four or five additional peaks selected from 16.8, 19.1, 21.6, 22.2 and 25.8 degrees 2-theta±0.2 degrees 2-theta. Alternatively crystalline form 2 can be characterized by an X-ray powder diffraction pattern having peaks at 5.8, 11.1, 13.7, 16.8, 17.3, 18.3, 19.1, 21.6, 22.2 and 25.8 degrees 2-theta±0.2 degrees 2-theta.

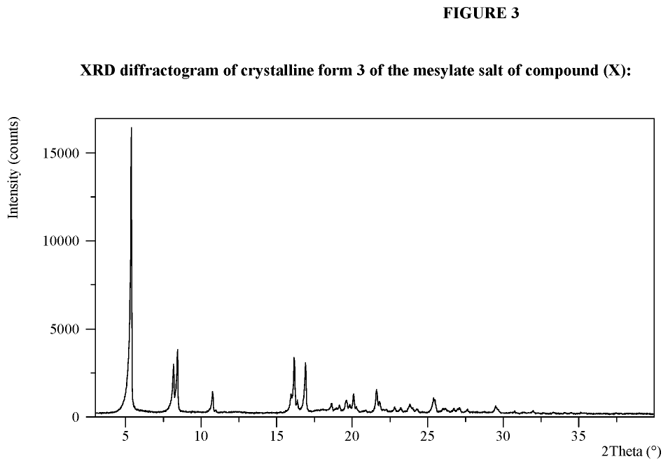

In yet another embodiment, the present invention discloses a crystalline form of the mesylate salt of compound (X); designated form 3. The crystalline form 3 may be characterized by data selected from one or more of the following: an X-ray powder diffraction pattern having peaks at 5.4, 8.4, 9.2, 10.8 and 16.2 degrees 2-theta±0.2 degrees 2-theta; an X-ray powder diffraction pattern substantially as depicted in FIG. 3; and combinations of these data. Crystalline form 3 can be further characterized by an X-ray powder diffraction pattern having peaks at 5.4, 8.4, 9.2, 10.8 and 16.2 degrees 2-theta±0.2 degrees 2-theta and also having any one, two, three, four or five additional peaks selected from 8.2, 16.9, 19.6, 20.1 and 21.6 degrees 2-theta±0.2 degrees 2-theta. Alternatively, crystalline form 3 can be characterized by an X-ray powder diffraction pattern having peaks at 5.4, 8.2, 8.4, 9.2, 10.8, 16.2 16.9, 19.6, 20.1 and 21.6 degrees 2-theta±0.2 degrees 2-theta.

The invention further provides the use of the crystalline forms of the mesylate salt of compound (X) as described herein in a process for preparing Zanubrutinib or any salt thereof.

In any aspect of the process disclosed therein, the conversion of the compound of formula (VII) to Zanubrutinib may be carried out by a process comprising:
(a) subjecting the compound (VII) to reduction and deprotection sequentially in any order, or simultaneously, to form a compound (VI):

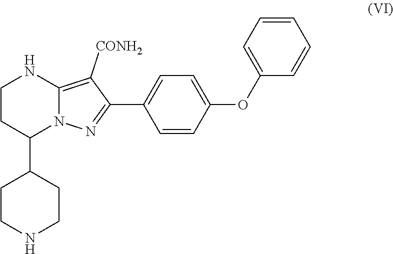 [SMILES: NC(=O)c1c(-c2ccc(Oc3ccccc3)cc2)nn2c1NCCC2C1CCNCC1]

b) chiral resolution of the compound (VI) to form a compound (III):

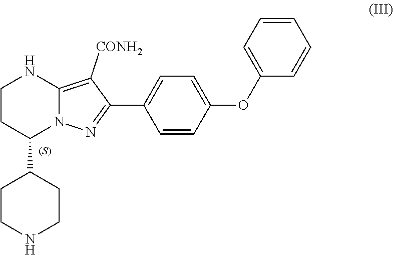

c) N-substitution of the compound (III) to form Zanubrutinib (I):

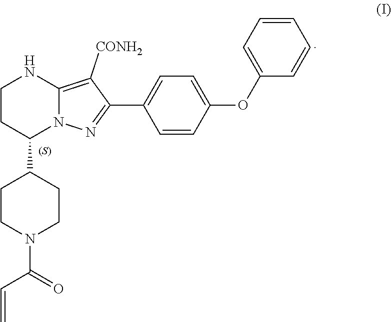

The above step (a) reduction and deprotection steps and may be carried out as previously described.

The chiral resolution step (b) can be carried out by diastereomeric crystallization using a chiral acid, optionally wherein the chiral acid is selected from the group consisting of: malic acid, mandelic acid, camphorsulfonic acid, tartaric acid, dibenzoyltartaric acid, di-p-toluoyltartaric acid, and optionally wherein the chiral acid is selected from the group consisting of: dibenzoyl-L-tartaric acid, dibenzoyl-D-tartaric acid, di-p-toluoyl-D-tartaric acid, di-p-toluoyl-L-tartaric acid, L-malic acid, D-malic acid, L-tartaric acid, D-tartaric acid, L-mandelic acid, D-mandelic acid, L-camphorsulfonic acid, or D-camphorsulfonic acid. Particularly, step (b) is carried out by diastereomeric crystallization using dibenzoyl-L-tartaric acid, di-p-toluoyl-L-tartaric acid or L-malic acid, optionally L-malic acid. The diastereomeric crystallisation may advantageously be carried out in a polar solvent, optionally a C₁₋₆ alcohol, water or mixtures of one or more of a C₁₋₆ alcohol and water, or wherein the polar solvent is methanol, ethanol, n-propanol, 2-propanol, n-butanol, 2-butanol, THF, acetonitrile, or their mixtures with water, or wherein the polar solvent is: methanol/water, ethanol, ethanol/water, n-propanol/water, 2-propanol/water, n-butanol/water, THF/water, or acetonitrile/water. Particularly n-propanol/water mixture in a v/v ratio of: 95:5 to 70:30, 90:10 to 80:20, or 88:12 to 82:18 or about 85:15 may be used, for example using dibenzoyl-L-tartaric acid as a resolving agent. Optionally, the diastereomeric crystallisation can be carried out by heating a solution comprising the compound (VI) with the chiral acid in the solvent, wherein the heating may be to a temperature of about 50° C. to about 100° C., about 60° C. to about 90° C., about 75° C. to about 85° C., or about 80° C. to about 83° C. The reaction is preferably allowed to cool slowly, to precipitate the chiral salt [i.e. compound (V)]. The chiral salt may be isolated in high purity. The purity may be further increased by one or more further crystallisations, for example using a mixture of alcohol/water, such as n-propanol/water.

In any aspect or embodiment described herein, the chiral salt (V) may be basified to form the compound of formula (III) which may be used in the next (N-substitution) step. Alternatively, the compound (V) can be employed directly in the N-substitution step, and basified prior to the reaction with the N-substitution reagent. The basification reaction results in a reaction mixture comprising compound (III). This reaction mixture, optionally after work-up, may be used directly in the N-substitution step, i.e. without isolating the chiral mediate. Hence, the N-substitution step as described below may be carried out by first basifying the chiral salt (V) in situ and conducting the N-substitution reaction without isolating the free base (III).

The N-substitution step (c) of the above process may be carried out by a procedure comprising:
(i) reacting the compound of formula (III) with compound (IV-A):

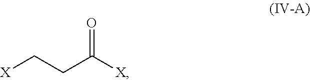

to prepare a compound of formula (II):

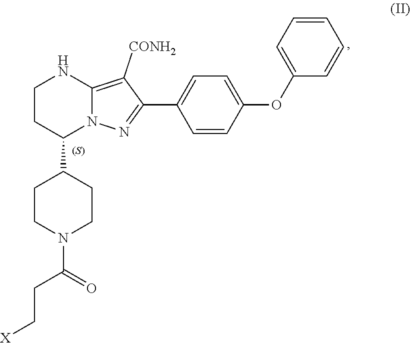

wherein X at either occurrence is the same or different and represents chloro or bromo, optionally wherein X at both occurrences is chloro (i.e. 3-chloropropionyl chloride); and
(ii) elimination to form Zanubrutinib (I).

As discussed above, according to any embodiment or aspect of the disclosure, the compound (III) may be formed by reaction of the chiral salt (V) with a base. The reaction of (V) with a base may be conducted to form a reaction mixture comprising compound (III) which can be used directly from this basification step (i.e. in situ) without isolating and/or without further purification. The above reaction of compound (III) with compound (IV-A), according to step (i) may be carried out in a polar solvent, optionally wherein the solvent comprises acetonitrile or a C₁₋₆ alcohol, or a mixture of acetonitrile and water, or a mixture of a C₁₋₆ alcohol and water, or wherein the solvent is a mixture of acetonitrile and water, or a mixture of n-propanol and water. Optionally, the solvent may be selected from the group consisting of acetonitrile, tetrahydrofuran, 2-methyl-tetrahydrofuran, methylethylketone, dichloromethane, ethyl acetate, toluene, acetone, C₁ to C₆ alcohol, optionally wherein the solvent is in a mixture with water, or wherein the solvent is a mixture of acetonitrile and water or a mixture of n-propanol and water.

The reaction of compound (III) with compound (IV-A) may be carried out in the presence of a base. Optionally, the reaction may be carried out in the presence of a polymerization inhibitor, polymerization retarders or radical scavengers. Suitable polymerization inhibitors include butylated hydroxytoluene, TEMPO, TEMPOL, p-phenylenediamines, phenothiazine and hydroxylamines such as hydroxyalkylhydroxylamine (HPHA) and diethylhydroxylamine (DEHA), quinones, and quinone methides. Suitable bases may be selected from alkali metal carbonates, alkali metal bicarbonates, or a tertiary C₁₋₄ alkyl amine. Optionally in any of the disclosed processes involving this step, the base may be sodium hydrogen carbonate.

The above reaction of compound (III) with compound (IV-A), according to step (i) may be carried out with cooling, for example to below room temperature. Optionally, the reaction is carried out at a temperature of about −10° C. to about 20° C., about -5° C. to about 15° C., about 0° C. to about 10° C., or about 5° C. to about 10° C.

The resulting N-substituted compound of formula (II), may be isolated by extraction and neutralisation of the reaction mixture with a base (typically an alkali metal carbonate or alkali metal hydrogen carbonate, such as sodium hydrogen carbonate, and extraction into a suitable solvent (e.g. 2-methyltetrahydrofuran). The resulting product may advantageously be isolated in high purity, avoiding the need to carry out further purification steps. Alternatively, compound of formula (II) may be used directly from the reaction mixture without isolating and/or without further purification.

In any of the herein discussed processes, the elimination step, whereby compound (II) is converted to Zanubrutinib (I), may be optionally carried out by base elimination, whereby compound (II) is reacted with a strong base. Particularly, compound (II) may be reacted with a base in a suitable solvent. The reaction may be carried out anhydrous and/or under inert conditions (e.g. under an atmosphere of nitrogen or argon), preferably both anhydrous and inert conditions. Optionally, the base may be selected from: potassium hydroxide, sodium hydroxide, 1,8-diazabicyclo[5.4.0]undec-7-ene (DBU), t-BuOK, NaOMe, and NaOEt. DBU or potassium hydroxide, particularly potassium hydroxide may be used. The base may be added dropwise to a solution of the starting compound (II), optionally at room temperature. The addition may be carried out over a period of: 0.5 to 3 hours, 0.5 to 2 hours or about 1 hour. The elimination reaction mixture may be heated at a temperature of about 10° C. to about 70° C., about 15° C. to about 65° C., about 20° C. to about 60° C., about 30° C. to about 55° C., about 40-50° C., or about 45-50° C. The solvent for the elimination step in any of the described processes may be optionally selected from the group consisting of 2-methyl-tetrahydrofuran (MeTHF), THF, ethyl acetate (EtOAc), acetonitrile, C₁-C₄ alcohol optionally methanol, ethanol, n-propanol, 2-propanol, t-butanol, or mixtures thereof with water; and optionally wherein the solvent is selected from a mixture of acetonitrile and water, or n-propanol/water. Optionally the elimination process may be conducted using potassium hydroxide and acetonitrile/water solvent. Following the reaction, the mixture may be cooled, optionally to room temperature, and neutralized with a mineral acid, such as hydrochloric acid. The resulting compound may be isolated by extraction and evaporation, and optionally recrystallised.

In the above described process wherein compound (VII) is converted to Zanubrutinib by: (a) reducing/deprotecting a compound (VII) to form a compound (VI), (b) chiral resolution to form the compound (III), and (c) N-substitution to form Zanubrutinib, the N-substitution step (c) may alternatively be carried out by a process comprising reacting the compound (III) with a compound (IV-B):

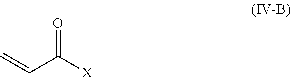

wherein X is chloro or bromo, optionally chloro.

The reaction of compound (III) with compound (IV-B) may be carried out in the presence of in a polar aprotic solvent, in the presence of a base. Optionally, the base may be selected from: potassium hydroxide, sodium hydroxide, 1,8-diazabicyclo[5.4.0]undec-7-ene. Optionally, the polar aprotic solvent may be selected from the group consisting of acetonitrile, tetrahydrofuran, 2-methyl-tetrahydrofuran, water, or mixtures thereof, optionally wherein the solvent is 2-methyl-tetrahydrofuran. The reaction may optionally be carried out at a temperature of about 10° C. to about 40° C., about 15° C. to about 30° C., about 20° C. to about 25° C.

A further aspect of the present disclosure provides a process for preparing Zanubrutinib comprising the steps of:
(A) reacting a compound (IX) with a compound (VIII) to form the compound (VII):

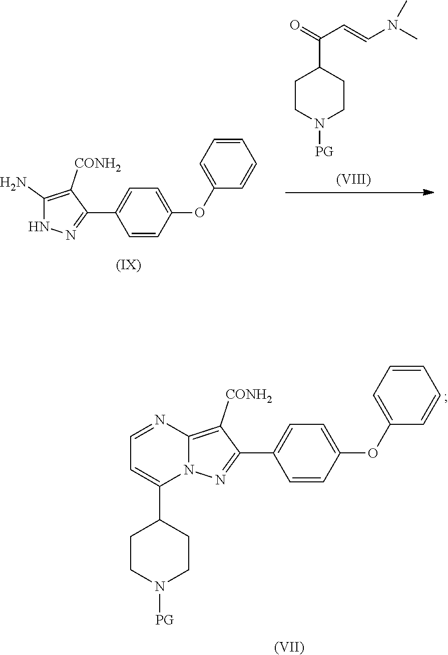

(B) reducing and deprotecting the compound (VII) according to form a compound (VI):

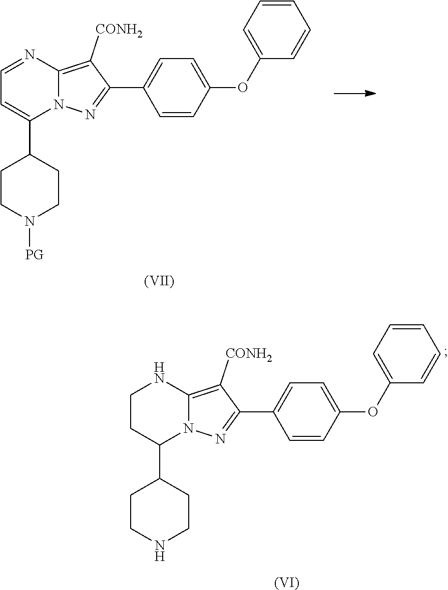

(C) chiral resolution of the compound (VI) to form a compound (III):

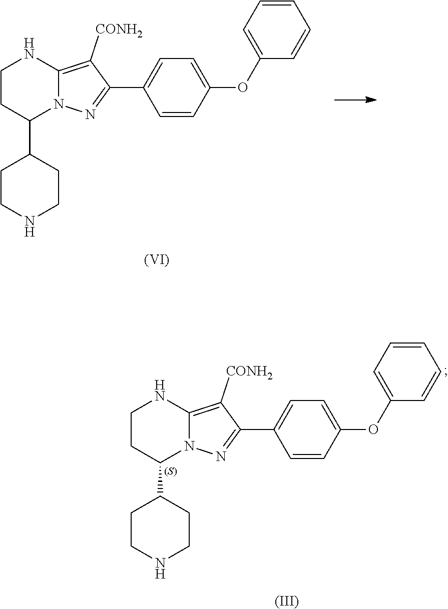

and
(D) N-substitution of the compound (III):

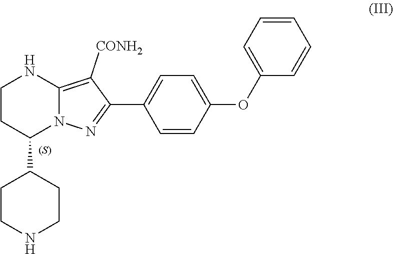

to form Zanubrutinib.

The coupling, reduction, deprotection and chiral resolution steps (A)-(D) of this process may be carried out as described in any of the disclosed embodiments. The starting material of formula (IX) may optionally be prepared according to any of the herein described embodiments.

Preferably according to any aspect or embodiment of the presently disclosed processes, step (C) is carried out by diastereomeric crystallisation using a chiral acid as discussed herein. After separation of the diastereomers, the desired chiral salt (V) may be basified to form the compound of formula (III) which may be used in step (D) without isolation of the compound (III). Hence, the N-substitution step (D) may be carried out by basifying the chiral salt (V) and, without isolating the free base (III), conducting the N-substitution reaction. In this case, the chiral salt (V) is first treated with a base in situ the resulting free base can be used without isolation in the N-substitution step.

The processes for preparing Zanubrutinib according to any aspect of the present disclosure may further comprise the step of converting Zanubrutinib into a salt thereof. The processes for preparing Zanubrutinib according to any aspect of the present disclosure may further comprise combining the Zanubrutinib with at least one pharmaceutically acceptable excipient to form a pharmaceutical formulation, optionally wherein the pharmaceutical formulation is an oral dosage form.

The present disclosure further provides a compound of Formula (VII):

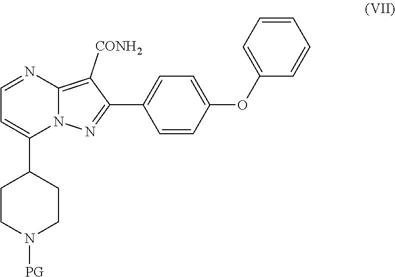

wherein PG is a protecting group, optionally acetyl, benzyl, methyl, benzoyl, toluoyl, methoxycarbonyl, ethoxycarbonyl, benzyloxycarbonyl, tert-butyloxycarbonyl, allyloxycarbonyl, 4-methoxybenzyl, para-methoxybenzylcarbonyl, 3,4-dimethoxybenzyoyl, propionyl, butyryl, phenylacetyl, phenoxyacetyl, trityl, 2,2,2-trichloroethoxycarbonyl, carbobenzoxy, 4-methoxybenzyloxycarbonyl, 9-fluorenylmethoxycarbonyl, 2-iodoethoxy-carbonyl, 4-methoxy-2,3,6-trimethylbenzenesulfonyl, methanesulfonyl, para-toluenesulfonyl, phenyl sulfonyl, trifluorocarbonyl, 2-trimethylsilylethoxycarbonyl or 4-nitrobenzenesulfonyl. According to an embodiment of the present disclosure, PG is selected from methoxycarbonyl, ethoxycarbonyl, benzyloxycarbonyl, or tert-butyloxycarbonyl, and optionally wherein PG is tert-butyloxycarbonyl and the compound (VII) has the formula:

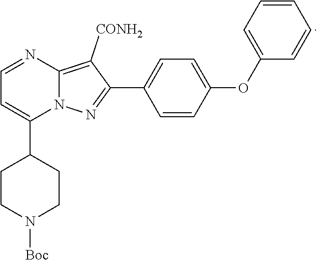 [SMILES: CC(C)(C)OC(=O)N1CCC(c2ccnc3c(C(N)=O)c(-c4ccc(Oc5ccccc5)cc4)nn23)CC1]

The present disclosure further provides a compound of Formula (II):

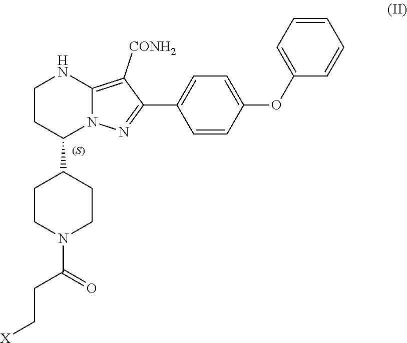

wherein X is chloro or bromo. Optionally, the compound (II) has the formula:

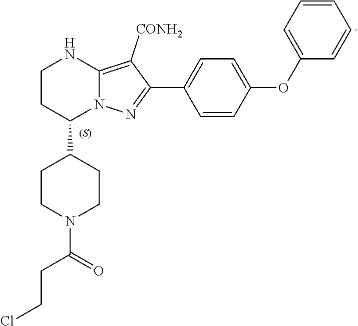

The present disclosure further provides the use of a compound of formula (VII), (II) as described herein for preparing Zanubrutinib or optionally a salt of Zanubrutinib.

An alternative process for preparing Zanubrutinib or a salt thereof is disclosed herein. The process involves reductive amination of the chiral intermediate of formula (XII) with the compound (IX) as described in any of the disclosure herein, to form the compound (XIII):

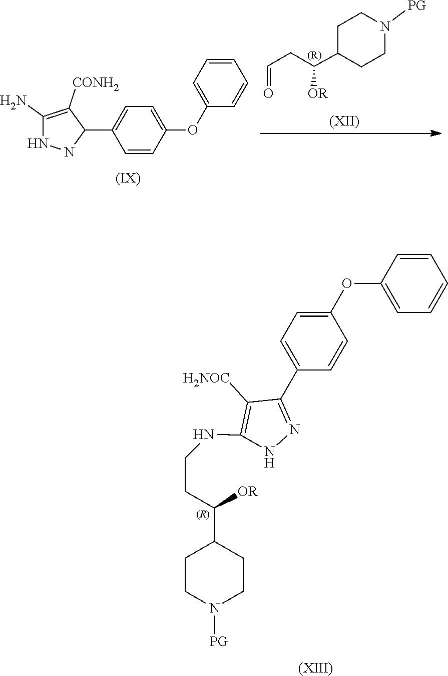

wherein PG is a protecting group (particularly Boc), and R represents H or a protecting group, optionally trialkylsilyl, and particularly tert-butyldimethylsilyl. The coupling reaction is carried out in the presence of a reducing agent, such as a borohydride, particularly sodium triacetoxyborohydride, optionally in the presence of an acid, particularly acetic acid (e.g. glacial acetic acid).

In yet a further aspect, the compound of formula (XIII) may be converted to Zanubrutinib by a process comprising:
cyclisation of the compound (XIII) to form a compound (XIV):

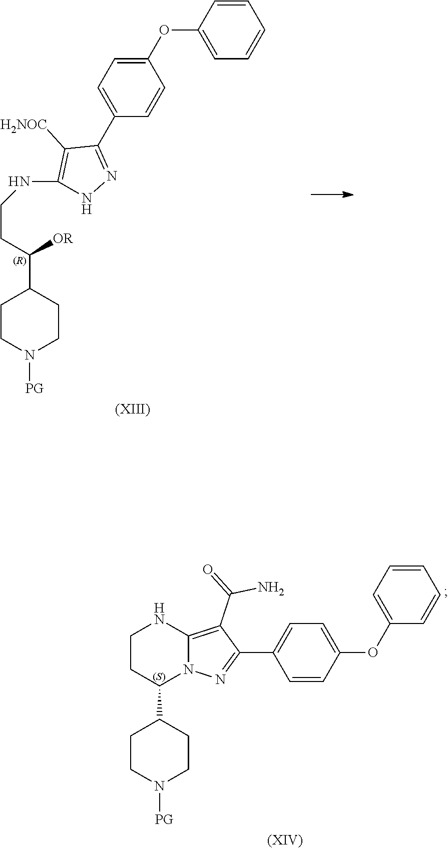

deprotection of the compound (XIV) to form the compound (III):

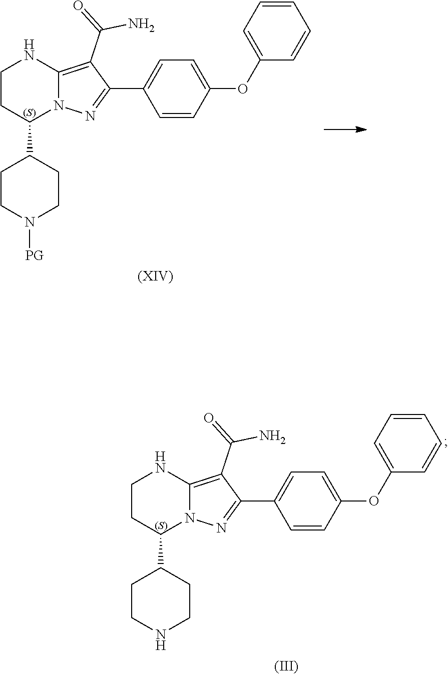

N-substitution of the compound of formula (III) to form Zanubrutinib:

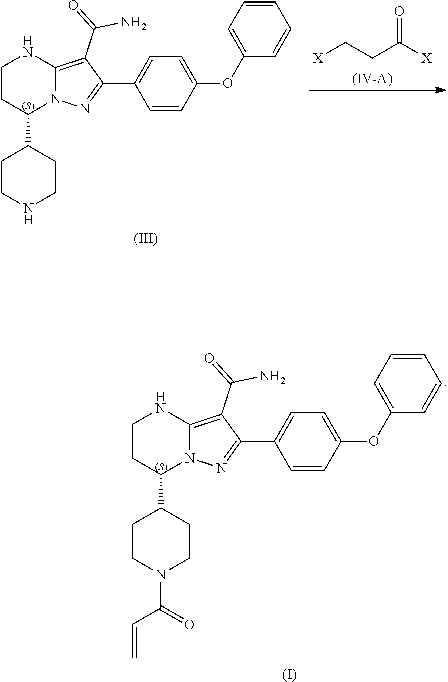

In the above conversion of the compound of formula (XIII) to Zanubrutinib, when R is H, the cyclisation reaction may be carried out by reaction of the compound (XIII) with a compound L-Y, wherein L can be tosyl or mesyl, particularly mesyl, and Y is chloro or bromo. Preferably the reaction is carried out using mesylchloride. The reaction may be carried out in the presence of a base, preferably an organic base, more particularly an amine, such as pyridine or piperidine or a mono-, di-, or trialkylamine. Alternatively when R in the compound (XIII) is protecting group such as trialkylsilyl (optionally tert-butyldimethylsilyl), the R group is removed, preferably by reaction with a quaternary ammonium fluoride compound, such as tert-n-butylammonium fluoride, and the cyclisation reaction may then be conducted by reaction with a compound L-Y as described above.

Following the cyclisation reaction, the compound (XIV) is deprotected to form the compound (III). The deprotection reaction may be carried out as described above for the compound of formula (VII) or (VII-A).

Thereafter, the compound of formula (III) may be converted to Zanubrutinib (I) as described in any of the embodiments disclosed herein.

The chiral intermediate (XII) as used in the above process may be prepared by a first step of enzymatic catalytic reduction of (XVIII):

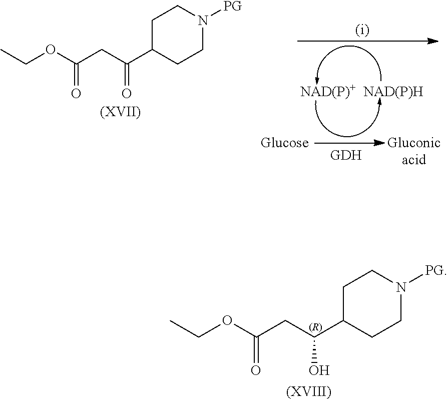

The process is preferably conducted using a ketoreductase enzyme, NADP (β-Nicotinamide adenine dinucleotide phosphate disodium salt) in a buffer solution, such as a 4-(2-hydroxyethyl)morpholine (HEM)/HCl buffer solution, and in the presence of glucose and glucodehydrogenase (GDH) enzyme. The process is preferably carried out at neutral pH, such as a pH of about 7±0.2. The reaction may be conducted at a temperature of about 20° C. to about 35° C., about 22° C. to about 32° C., about 25° C. to about 30° C., or about 27° C. to about 29° C. or about 28° C. Suitable ketodreductase enzymes include PRO-KREDP-283, PRO-KREDP-281, PRO-KREDP-640, PRO-KREDP-178, PRO-KREDP-360 obtainable from Prozomix, or ES-KRED-241, ES-KRED-239 obtainable from SyncoZymes. This process enables the production of the compound (XVIII) having a high optical purity.

The compound of formula (XVIII) is then subjected to one of the following processes:
reduction of the compound (XVIII) to form the compound (XIX) wherein R is H; and oxidation of the compound (XIX), to form a compound of formula (XII) wherein R is H; or
protecting the hydroxy group of the compound (XVIII) with a protecting group P1 to form the compound (XVIII-A); reduction of the compound of formula (XVIII-A) to form the compound of formula (XIX) wherein R is P1; reduction of the compound (XVIII-A) to form the compound (XIX) wherein R is P1; and oxidation of the compound of (XIX) to form a compound of formula (XII) wherein R is P1

The reduction of the compound (XIX) wherein R is H or P1 may be carried out using a hydride reducing agent, optionally sodium bis(2-methoxyethoxy)aluminum hydride. The reaction may be carried out in a polar aprotic solvent, such as THF. The reaction may optionally be carried out under an inert atmosphere. Optionally the reaction is carried out under cooling, typically: −10° C. to 15° C., −5° C. to 10° C. or −2° C. to 2° C., or about 0° C.

The oxidation of the terminal hydroxyl group in the compound (XIX) wherein R is H or P1, to an aldehyde to form the compound (XII), is carried out using an oxidizing agent, such as sodium hypochlorite, preferably using an oxidation catalyst, particularly 2,2,6,6-tetramethylpiperidine-l-oxyl radical (TEMPO). The reaction may be carried out in the presence of a base and a bromide source, such as potassium bromide. Optionally the reaction is carried out under cooling, typically: −10° C. to 15° C., −5° C. to 10° C. or −2° C. to 2° C., or about 0° C.

The step of protecting the hydroxy group of the compound (XVIII) with a protecting group P1 to form the compound (XVIII-A) may be carried out by reaction with a reagent P1-Y, where P1 is preferably trialkylsilyl, particularly tert-butyldimethylsilyl, and Y is chloro or bromo, particularly chloro. The reaction may optionally be carried out in the presence of a base, particularly an organic base, such as imidazole.

The present disclosure further provides compounds of formula (XIII) and (XIV), and their use in the synthesis of Zanubrutinib or a salt thereof. Disclosed are compounds of formula (XII):

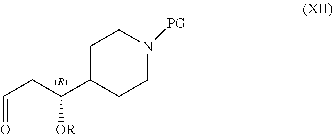

wherein R is H or trialkylsilyl, preferably tert-butyldimethylsilyl; and PG is a protecting group as defined in any of the disclosure herein. In embodiments R is H and PG is Boc, or R is tert-butyldimethylsilyl and PG is Boc.

Also disclosed are compounds of formula (XIII):

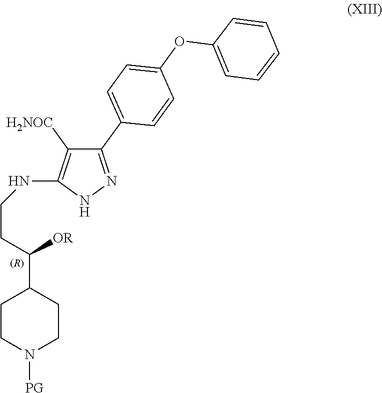

wherein R is H or trialkylsilyl, preferably tert-butyldimethylsilyl; and PG is a protecting group as defined in any of the disclosure herein. In embodiments R is H and PG is Boc, or R is tert-butyldimethylsilyl and PG is Boc.

The disclosure also provides the use of a compound of formula (XVII), (XVIII), (XVIII-A), (XIX), (XII), (XIII), or (XIV) in a process for the preparation of Zanubrutinib, or a salt thereof.

According to any aspect or embodiment of the present disclosure, the compounds (XVII), (XVIII), (XVIII-A), (XIX), (XII), (XIII), or (XIV) may be substantially pure, i.e. having a purity of: greater than about 95%, greater than about 97%, greater than about 99%, or greater than about 99.3%, or greater than about 99.8% (by weight).

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### ANALYTICAL METHODS

X-ray powder diffraction method: X-ray diffraction was performed on X-Ray powder diffractometer. After being powdered using mortar and pestle, samples were applied directly on a silicon plate holder. The X-ray powder diffraction pattern was measured with a Philips X'Pert PRO X-ray powder diffractometer, equipped with a Cu irradiation source=1.5418 \acute{Å} (\acute{Å}ngström), X′Celerator (2.022° 2θ) detector. Scanning parameters: angle range: 3-40 deg., step size 0.0167, time per step 37 s, continuous scan. The accuracy of peak positions was defined as +/− 0.2 degrees due to experimental differences, such a differences in instrumentation and/or sample preparation.

NMR: NMR Spectra were taken on a Bruker Avance 400 MHz (400 MHz of frequency for ¹H NMR and 100.61 MHz of frequency ¹³C NMR) at 299K.

Mass Spectra: The ES-MS spectra were taken on ABSCIEX 4600 Q TOF instrument.

### EXAMPLES

### Example 1A

### Synthesis of 5-amino-3-(4-phenoxyphenyl)-1H-pyrazole-4-carboxamide methanesulfonate)

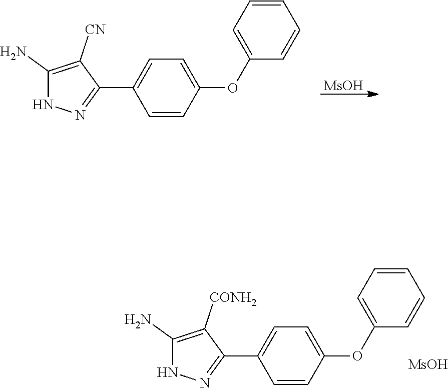

5-Amino-3-(4-phenoxyphenyl)-1H-pyrazole-4-carbonitrile (20 g, 72,388 mmol) was charged into the 250-mL reactor together with 100 mL (5 volumes) of methanesulfonic acid and the reaction mixture was warmed up to 60° C. until dissolution occurred. The obtained solution was stirred for 18 hours, cooled to 20-25° C. and 70 mL (3.5 volumes) of water was added drop wise. Slightly turbidous solution was allowed to crystallize for 1 hour and another portion of water (80 mL, 4 volumes) was added drop wise during 30 minutes, which resulted with formation of a white suspension. The mixture was stirred for another 2 hours at 20-25° C. and filtered off. The crystals were washed with 2×2 volumes and 1×1 volume of water and dried for 10 hours at 50° C. and 10 mbar to obtain 25.4 g (90%) of 5-amino-3-(4-phenoxyphenyl)-1H-pyrazole-4-carboxamide methanesulfonate with 95.11% purity. The solid was analyzed by XRD (FIG. 1).

### Example 1B

### Synthesis of 5-amino-3-(4-phenoxyphenyl)-1H-pyrazole-4-carboxamide methanesulfonate)

5-amino-3-(4-phenoxyphenyl)-1H-pyrazole-4-carbonitrile (20 g) was charged in a 1 L reactor and dissolved in methanesulfonic acid (120 ml) at 80° C. The reactor was then purged with nitrogen and stirred with a nitrogen flow for 3 hours. The mixture was then cooled to 5° C. and water (70 ml) was added dropwise while maintaining the temperature below 30° C. The mixture was seeded and water (20 ml) was added dropwise to start the crystallization. The mixture was stirred for an hour at room temperature and water (78 ml) was then added dropwise. A white suspension formed which was stirred over 72 hours at room temperature. The mixture was then filtered under nitrogen and the crystals were washed two times with water (40 ml) and once more with 10 ml. The solids were dried for 10 hours (50° C., 10 mbar) and analyzed by XRPD-FIG. 2 (Mesylate salt of compound (X)-form 2).

### Example 1C

### Synthesis of 5-amino-3-(4-phenoxyphenyl)-1H-pyrazole-4-carboxamide methanesulfonate)

In the 1L reactor water (550 mL) was charged and cooled to 10-15° C. 50 mL of solution 5-amino-3-(4-phenoxyphenyl)-1H-pyrazole-4-carboxamide in methanesulfonic acid (154 g/L; 50 mL; 7.7 g; 26.1 mmol) was added over 20 minutes period while stirring the mixture. The temperaure of the suspension was kept below 25° C. Suspension was stirred additional 15 minutes, filtrated and washed with water (2×10 mL). Wet crystals were dried at 50° C. (10 mbar) till constant mass was obtained. The solid was analyzed by XRD-FIG. 3 (Mesylate salt of compound (X)-form 3).

### Example 2A

### Synthesis of 5-amino-3-(4-phenoxyphenyl)-1H-pyrazole-4-carboxamide

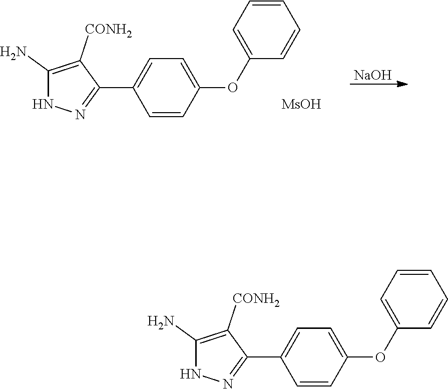 [SMILES: "MsOH" ; NC(=O)c1c(-c2ccc(Oc3ccccc3)cc2)n[nH]c1N ; "NaOH" ; NC(=O)c1c(-c2ccc(Oc3ccccc3)cc2)n[nH]c1N]

5-Amino-3-(4-phenoxyphenyl)-1H-pyrazole-4-carboxamide methanesulfonate (25 g, 64.035 mmol) was charged into the 250-mL reactor together with 250 mL (10 volumes) of water. The pH of the obtained suspension was adjusted to 7 by 2.5M NaOH (approx. 26 mL). The obtained mixture was stirred at 20-25° C. for 1 hour and filtered off. The crystals were washed with 2×2 volumes of water and dried for 10 hours at 50° C. and 10 mbar to obtain 19.0 g (100%) of 5-amino-3-(4-phenoxyphenyl)-1H-pyrazole-4-carboxamide with 96.53% purity.

### Example 2B

### Synthesis of 5-amino-3-(4-phenoxyphenyl)-1H-pyrazole-4-carboxamide

A mixture of 5-amino-3-(4-phenoxyphenyl)-1H-pyrazole-4-carbonitrile (65 g) and methanesulfonic acid (390 ml) was heated to 60° C. and stirred overnight. The mixture was then cooled to room temperature and water (357,5 ml) was added dropwise to the solution. Obtained suspension was stirred for one hour. Additional water (188,5 ml) was added dropwise and the mixture was stirred for another hour. Obtained off-white suspension was filtered and washed with water (2*130 ml and 1*65 ml). The solids were then charged back into the reactor, suspended in water (819 ml) and pH value was adjusted to 11 using 10M NaOH solution. The white slurry was filtered, washed with water (2*162,5 ml) and dried in oven under vacuum (50° C., 10 mbar) for ten hours to obtain 5-amino-3-(4-phenoxyphenyl)-1H-pyrazole-4-carboxamide. The solid was analyzed by)(RFD-FIG. 4.

### Example 3A

### Synthesis of tert-butyl 4-(3-carbamoyl-2-(4-phenoxyphenyl)pyrazolo11,5-alpyrimidin-7-yl)piperidine-l-carboxylate

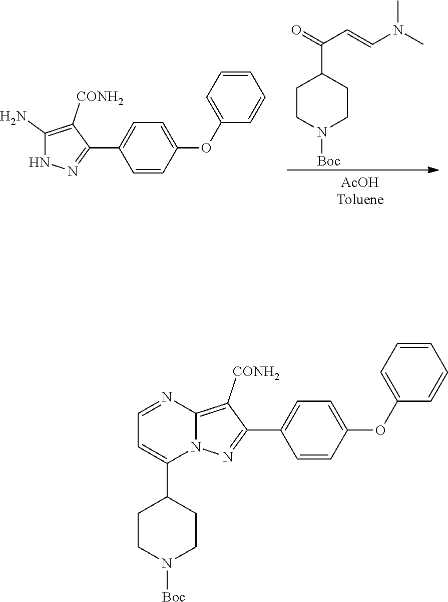

5-Amino-3-(4-phenoxyphenyl)-1H-pyrazole-4-carboxamide (18 g, 61.160 mmol) was charged into the 250-mL reactor together with 144 mL of toluene (8 volumes), and tent-butyl 4-(3-(dimethylamino)acryloyl)piperidine-l-carboxylate (20.7 g, 73.392 mmol, 1.20 molEq) was added followed by glacial acetic acid (9 mL, 0.157 mol, 2.57 molEq, 0.5 volumes). The obtained mixture was warmed up to 95° C. Obtained solution was stirred for 12 hours at 95° C. and at 20-25° C. for additional 10 hours. The obtained suspension was cooled to 0-5° C., stirred for 1 hour and filtered off. White crystals were washed with 2×2 volumes of toluene yielding 24 g (77%) of tent-butyl 4-(3-carbamoyl-2-(4-phenoxyphenyl)pyrazolo[1,5-a]pyrimidin-7-yl)piperidine-1- carboxylate with 97.99% purity.

### Example 3B

### Synthesis of tert-butyl 4-(3-carbamoyl-2-(4-phenoxyphenyl)pyrazolo[1,5-a]pyrimidin-7-yl)piperidine-1-carboxylate

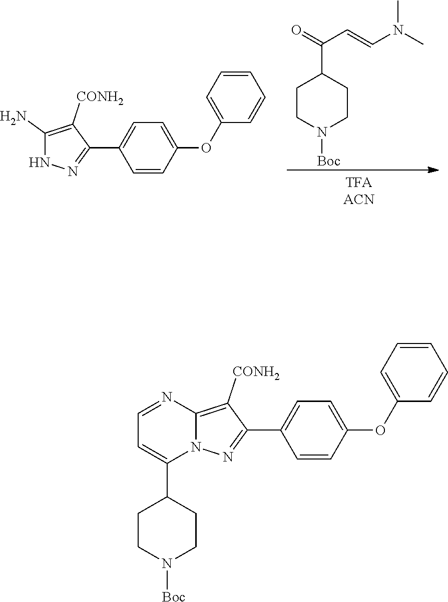

5-Amino-3-(4-phenoxyphenyl)-1H-pyrazole-4-carboxamide (40 g, 0.136 mol) was charged into the 1-L reactor together with 320 mL of acetonitrile (8 volumes), and tert-butyl 4-(3-(dimethylamino)acryloyl)piperidine-1-carboxylate (40.3 g, 0.143 mol, 1.05 molEq) was added, forming a yellow suspension. A solution of trifluoroacetic acid (11.45 mL, 0.150 mol, 1.10 molEq) in 80 mL of acetonitrile (2 volumes) was added dropwise into the reaction mixture in about 15 minutes at 20-25° C. The obtained mixture was warmed up to 40° C. Obtained solution was stirred for 3 hours at 40° C. and at 20-25° C. for additional 1 hour. The obtained suspension was filtered off and the pale yellow crystals were washed with 2×2 volumes of acetonitrile yielding 63.4 g (91%) of tert-butyl 4-(3-carbamoyl-2-(4-phenoxyphenyl)pyrazolo[1,5-a]pyrimidin-7- yl)piperidine-1-carboxylate with 98.45% purity.

### Example 3C

### Synthesis of tert-butyl 4-(3-carbamoyl-2-(4-phenoxyphenyl)pyrazolo[1,5-a]pyrimidin-7-y1)piperidine-1-carboxylate

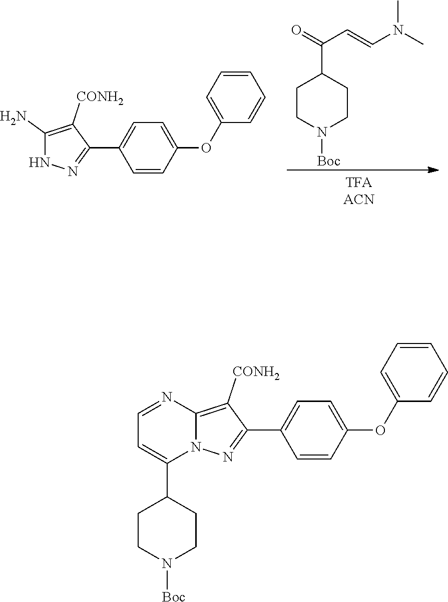

5-amino-3-(4-phenoxyphenyl)-1H-pyrazole-4-carboxamide (59 g) was charged in a 1 L reactor followed by tert-butyl (E)-4-(3-(dimethylamino)acryloyl)piperidine-1-carboxylate (57,1 g) and acetonitrile (472 ml). The yellow suspension was stirred at ambient temperature, and a solution of trifluoroacetic acid (16,2 ml) in acetonitrile (118 ml) was added dropwise in about 15 minutes at 20-25° C. The mixture was then heated to 40° C. and stirred for about 10 hours (until 5-amino-3-(4-phenoxyphenyl)-1H-pyrazole-4-carboxamide area % by HPLC was less than 1%). The yellow suspension was then cooled to 20° C. and stirred for 1 hour before filtration. The pale yellow crystals of tert-butyl 4-(3-carbamoyl-2-(4-phenoxyphenyl)pyrazolo[1,5-a]pyrimidin-7-yl)piperidine-1-carboxylate were washed twice with acetonitrile (2×118 ml), dried for 10 h (50° C. and 10 mbar) and analyzed by XRPD-FIG. 5.

### Example 4A

### Synthesis of 244-phenoxyphenyl)-7-(piperidin-4-yl)-4,5,6,7-tetrahydropyrazolo[1,5-a] pyrimidine-3-carboxamide racemic

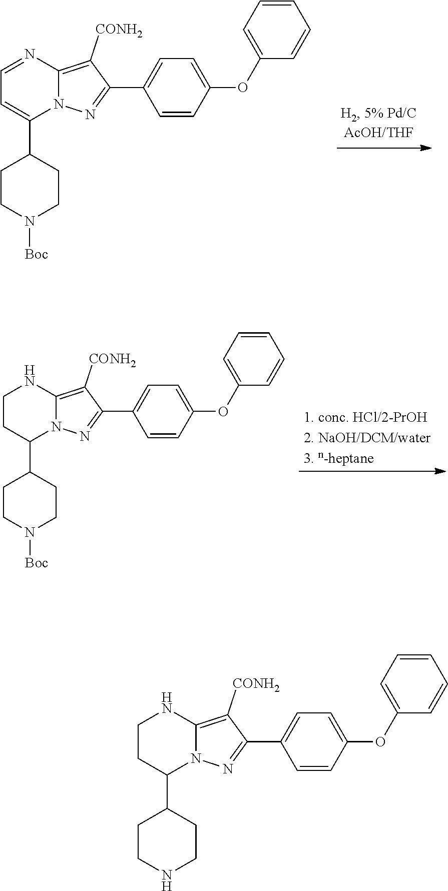 [SMILES: CC(C)(C)OC(=O)N1CCC(c2ccnc3c(C(N)=O)c(-c4ccc(Oc5ccccc5)cc4)nn23)CC1 ; CC(C)(C)OC(=O)N1CCC(C2CCNc3c(C(N)=O)c(-c4ccc(Oc5ccccc5)cc4)nn32)CC1 ; NC(=O)c1c(-c2ccc(Oc3ccccc3)cc2)nn2c1NCCC2C1CCNCC1]

tert-Butyl 4-(3-carbamoyl-2-(4-phenoxyphenyl)pyrazolo[1,5-a]pyrimidin-7-yl)piperidine-1-carboxylate (23 g, 44.783 mmol) was charged into the 600-mL autoclave together with 460 mL of tetrahydrofuran (20 volumes), 2.56 mL of acetic acid (44.783 mmol, 1 molEq) and 2.30 g of 5% Pd/C (0.10 mass %) and the mixture was subjected to hydrogenation at 60° C. and 2 bar of hydrogen for 24 hours. Reaction mixture was filtered off and the filtrate was evaporated to remove the solvent yielding tent-butyl 4-(3-carbamoyl-2-(4-phenoxyphenyl)-4,5,6,7-tetrahydropyrazolo[1,5-a]pyrimidin-7-yl)piperi-dine-1-carboxylate which undergoes in situ deprotection and neutralization as follows: tent-Butyl 4-(3-carbamoyl-2-(4-phenoxyphenyl)-4,5,6,7-tetrahydropyrazolo[1,5-a]pyrimidin-7-yl)piperidine-1-carboxylate (16.9 g, 32.649 mmol) was dissolved in 340 mL of 2-propanol (20 volumes) and 34 mL of 36% HCl (0.402 mmol, 12.3 molEq) was added and the reaction mixture was warmed up to 60° C. The reaction mixture was stirred for 1.5 hours and cooled to room temperature. Water (85 mL, 5 volumes) was added and pH was adjusted to 12.6 with 12 M NaOH (38mL). 2-Propanol was removed by distillation and dichloromethane (170 mL, 10 volumes) was added followed by separation of layers. Organic layer was dried over Na₂SO₄, filtered off and subjected to solvent exchange with n-heptane (170 mL, 10 volumes). Obtained precipitate was filtered off, the solid was washed with 2×2 volumes of n-heptane and dried for 8 hours at 50° C. yielding 3.4 g (25%) of 2-(4-phenoxyphenyl)-7-(piperidin-4-y1)-4,5,6,7-tetrahydro-pyrazolo[1,5-c]pyrimidine-3-carboxamide racemic with 99.32% purity. Mother liquor was concentrated in vacuo and precipitated from n-heptane (230 mL, 10 V/w) yielding additional 9.1 g of 2-(4-phenoxyphenyl)-7-(piperidin-4-yl)-4,5,6,7-tetrahydro-pyrazolo[1,5-c]pyrimidine-3-carboxamide (67%) with 95.34% purity (total yield 91%).

### Example 4B

### Synthesis of 2-(4-phenoxyphenyl)-7-(piperidin-4-yl)-4,5,6,7-tetrahydropyrazolo[1,5-a] pyrimidine-3-carboxamide racemic

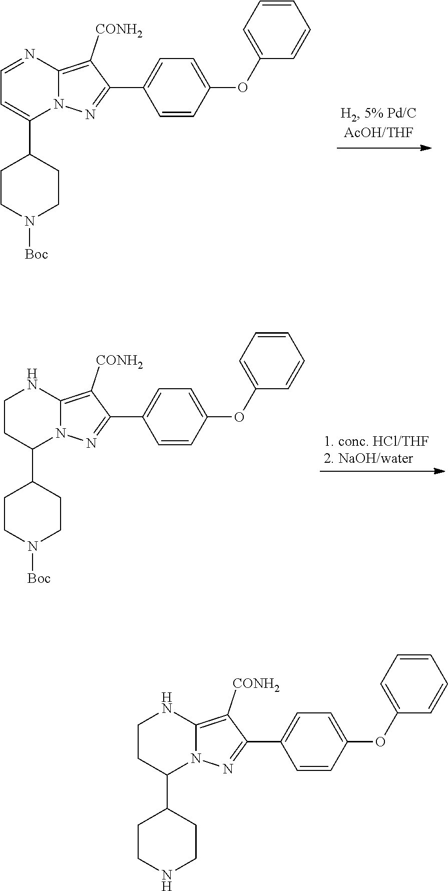 [SMILES: CC(C)(C)OC(=O)N1CCC(c2ccnc3c(C(N)=O)c(-c4ccc(Oc5ccccc5)cc4)nn23)CC1 ; CC(C)(C)OC(=O)N1CCC(C2CCNc3c(C(N)=O)c(-c4ccc(Oc5ccccc5)cc4)nn32)CC1 ; NC(=O)c1c(-c2ccc(Oc3ccccc3)cc2)nn2c1NCCC2C1CCNCC1]

tert-Butyl 4-(3-carbamoyl-2-(4-phenoxyphenyl)pyrazolo[1,5-a]pyrimidin-7-yl)piperidine-1-carboxylate (160 g, 0.312 mol) was charged into the 5-L autoclave together with 3.2 L of tetrahydrofuran (20 volumes), 17.8 mL of acetic acid (0.312 mol, 1 molEq) and 16.0 g of 5% Pd/C (0.10 mass %) and the mixture was subjected to hydrogenation at 60° C. and 6 bar of hydrogen for 24 hours with vigorous stirring. Reaction mixture was filtered off and the filtrate with tent-butyl 4-(3-carbamoyl-2-(4-phenoxyphenyl)-4,5,6,7-tetrahydropyrazolo[1,5-a]pyrimidin-7-yl)piperi-dine-1-carboxylate was subjected to in situ deprotection and neutralization. The filtrate was charged in a 6 L reactor and 160 mL (1.90 mol; 6.1 molEq) of concentrated hydrochloric acid was added. The clear colorless solution was heated to 50° C. and stirred for 1 hour. 3.04 L (19 volumes) of water was added and THF was distilled off until only water remained and the mixture slowly crystallized. pH value was adjusted to 12 using 10 M NaOH at ambient temperature. The obtained white suspension was heated to 90° C. followed by pH correction. The mixture was stirred at 90° C. for 1 hour and cooled to ambient temperature. White crystals of racemic 2-(4-phenoxyphenyl)-7-(piperidin-4-yl)-4,5,6,7-tetrahydropyrazolo[1,5 -a] pyrimidine-3-carboxamide were filtered off and washed two times with 320 mL of water (2 volumes) and dried for 10 hours at 50° C. and 10 mbar. The crystals were left overnight in an open container until the crystals reached the equilibrium water content of 7.5-8.0%, which corresponds to 2-(4-phenoxyphenyl)-7-(piperidin-4-yl)-4,5,6,7-tetrahydropyrazolo[1,5 -a] pyrimidine-3-carboxamide dihydrate, in 95% yield and 98.45% purity.

### Example 5A

### Resolution of 2-(4-phenoxyphenyl)-7-(piperidin-4-yl)-4,5,6,7-tetrahydropyrazolo [1,5-a] pyrimidine-3-carboxamide

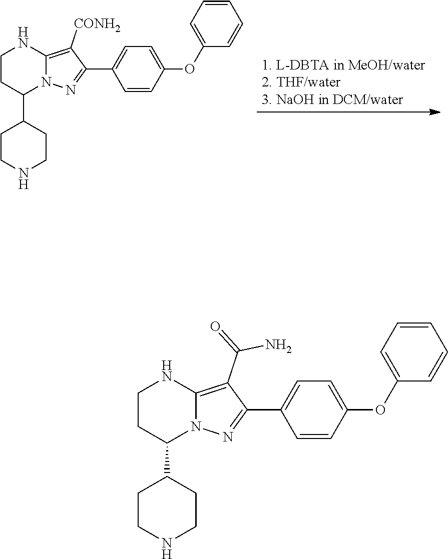 [SMILES: NC(=O)c1c(-c2ccc(Oc3ccccc3)cc2)nn2c1NCCC2C1CCNCC1 ; NC(=O)c1c(-c2ccc(Oc3ccccc3)cc2)nn2c1NCC[C@H]2C1CCNCC1]

The resolution of 2-(4-phenoxyphenyl)-7-(piperidin-4-yl)-4,5,6,7-tetrahydropyrazolo [1,5-a]pyrimidine-3-carboxamide may be carried out according to process known in the art; using the method from WO 2018/033853, Ex. 10, Chiral resolution of BG-12A, Page 41-42, according to which (S)-2-(4-phenoxyphenyl)-7-(piperidin-4-yl)-4,5,6,7-tetrahydropyrazolo [1,5-a]pyrimidine -3-carboxamide was isolated in total yield 30% and 96.5% enantiomeric excess (e e)

### Example 5B

### Resolution of 2-(4-phenoxyphenyl)-7-(piperidin-4-yl)-4,5,6,7-tetrahydropyrazolo [1,5-a] pyrimidine-3-carboxamide

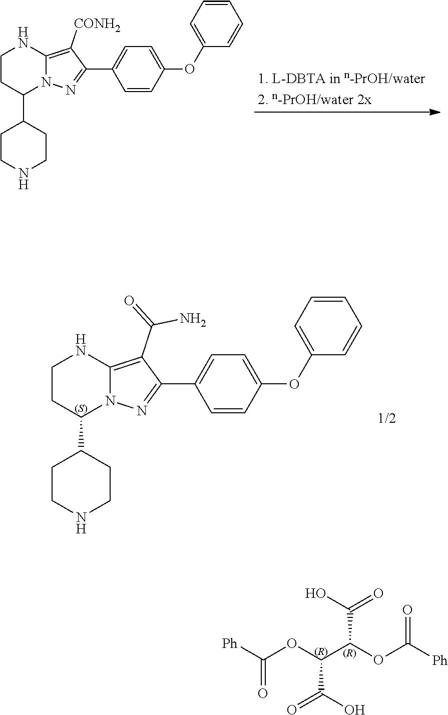

4 g of 2-(4-phenoxyphenyl)-7-(piperidin-4-yl)-4,5,6,7-tetrahydro-pyrazolo[1,5-c]pyrimidine-3-carboxamide, racemic, was charged with n-propanol/water (85/15 V/V; 120 mL) into the 250-mL 3-neck round-bottom flask equipped with mechanical stirrer, thermometer and condenser. The resulting suspension was heated up to 82° C. Above 60° C. the suspension turned into the solution. At 80-83° C., 1.17 g of L-DBTA (3.109 mmol, 0.35 molEq), previously dissolved in n-propanol/water (85/15 V/V; 20 mL), was added drop-wise (during 5 min) into the solution of racemic 2-(4-phenoxyphenyl)-7-(piperidin-4-yl)-4,5,6,7-tetrahydro-pyrazolo[1,5 -a] pyrimidine-3- carboxamide, racemic. The resulting solution was cooled down to 66° C. and seeded with (S)-2-(4-phenoxyphenyl)-7-(piperidin-4-yl)-4,5,6,7-tetrahydro-pyrazolo[1,5-c]pyrimidine-3- carboxamide×½L-DBTA (13 mg). Crystallisation started soon after seeding. Rare white suspension was stirred for 1 h at 65-70° C., then it was slowly cooled down to 25° C. by turning off the heater but leaving the flask in warm oil bath. At 25° C. the suspension was stirred for 10 hours and filtered off. White crystals were washed with n-propanol/water (85/15 V/V; 2×8 mL). The crystals were dried at 10 mbar and 50° C. for 3 hours yielding 2 g of ((S)-4-(3-carbamoyl-2-(4-phenoxyphenyl)-4,5,6,7-tetrahydropyrazolo[1,5-a]pyrimidin-7-yl)piperidin-l-ium) ((2R,3R)-2,3-bis(benzoyloxy)-succinate) hemi-salt (35.8% yield) of 94.28% chiral purity (ee 88.56%). The obtained salt was recrystallized two times from n-propanol/water in a way that it was suspended in 30 volumes of n-propanol/water (1/1 V/V and warmed up to 85° C. until dissolution. Water (30 volumes) was added drop-wise during 30 minutes with maintaining the temperature between 80° C. and 85° C. The solution was then cooled down to 72° C. where it is seeded with (S)-2-(4-phenoxyphenyl)-7-(piperidin-4-yl)-4,5,6,7-tetrahydro-pyrazolo[1,5-c]pyrimidine-3-carboxamide×½L-DBTA (0.25 mass %). The crystallization started immediately forming rare white suspension. The suspension was stirred at 73-78° C. for 1 hour, then slowly cooled down to 25° C. by turning off the heater but leaving the flask in warm oil bath. At 25° C. the propanol was stirred for 10 hours and filtered off. White crystals were washed with n-PrOH/water (1/3 V/V, 3×2 volumes) and dried at 10 mbar and 50° C. for 3 h. The yield after the 1^{st} recrystallization was 81% and chiral purity 99.32% (ee 98.64%). The yield after the 2^{nd} recrystallization was 87% and chiral purity 99.94% (ee 99.88%).

### Example 5C

### Neutralization of (S)-244-phenoxyphenyl)-7-(piperidin-4-yl)-4,5,6,7-tetrahydropyrazolo 11,5-al pyrimidine-3-carboxamide L-DBTA salt

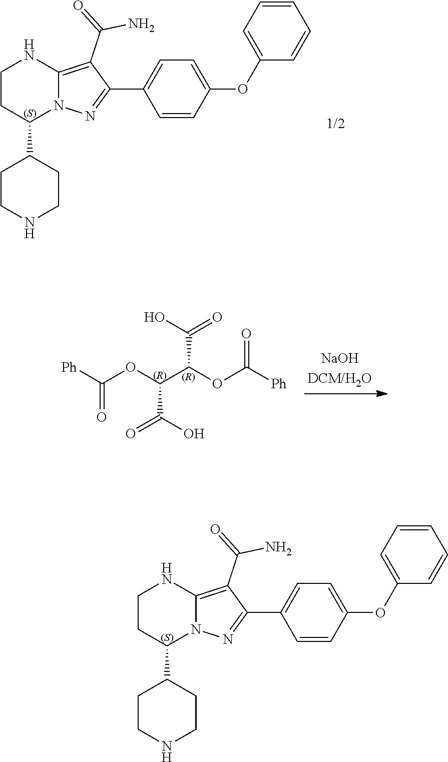

1.65 g of (S)-2-(4-phenoxyphenyl)-7-(piperidin-4-yl)-4,5,6,7-tetrahydro-pyrazolo[1,5-c]pyrimidine-3-carboxamide, L-DBTA hemi salt, was charged with a mixture of DCM (165m1) and water (165m1) into the 250-mL 3-neck round-bottom flask equipped with mechanical stirrer, thermometer and condenser. The resulting solution was treated with 2M NaOH aq. solution until pH is adjusted to >12. Layers were separated and aqueous layer was extracted with DCM (50 volumes). After separation of layers, two organic layers were joined together, washed with water (50 volumes) and evaporated until the white solid foam was obtained (99.8% yield). The chiral purity remained unchanged.

### Example 6

### Synthesis of 7-(1-acryloylpiperidin-4-yl)-2-(4-phenoxyphenyl)-4,5,6,7-tetrahydropyrazolo[1,5-a]pyrimidine-3-carboxamide from 2-(4-phenoxyphenyl)-7-(piperidin-4-yl)-4,5,6,7-tetrahydropyrazolo [1,5-a]pyrimidine-3-carboxamide

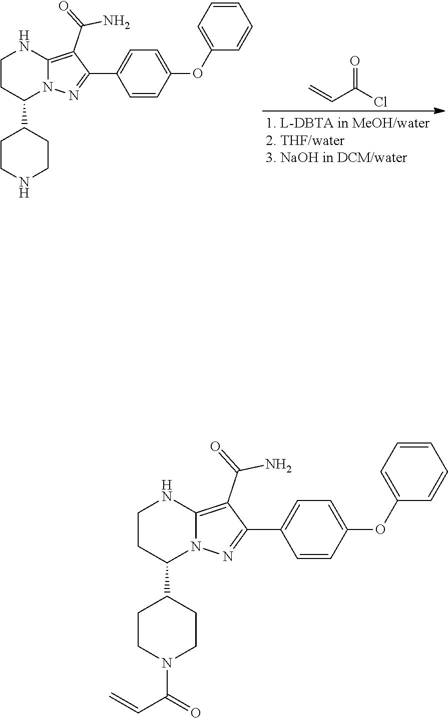

The preparation of 7-(1-acryloylpiperidin-4-yl)-2-(4-phenoxyphenyl)-4,5,6,7-tetrahydro pyrazolo[1,5-c]pyrimidine-3-carboxamide from 2-(4-phenoxyphenyl)-7-(piperidin -4-yl)-4,5,6,7-tetrahydropyrazolo[1,5-c]pyrimidine-3-carboxamide may be carried out according to process known in the art; using the method from WO 2018/033853, Ex. 1, Step 15, Synthesis of (S)-7-(1-acryloylpiperidin-4-yl)-2-(4-phenoxy phenyl)-4,5,6,7-tetrahydropyrazolo[1,5-c]pyrimidine-3-carboxamide (Compound 1, alternative method), Page 24, according to which crude 7-(1-acryloylpiperidin-4-yl)-2-(4-phenoxyphenyl)-4,5,6,7-tetrahydro pyrazolo[1,5-a]pyrimidine-3-carboxamide was isolated in 75% yield and 94.23% purity.

### Example 7A

### Synthesis of 7-(1-(3-chloropropanoyl)piperidin-4-yl)-2-(4-phenoxyphenyl)-4,5,6,7-tetrahydropyrazolo[1,5-a]pyrimidine-3-carboxamide racemic

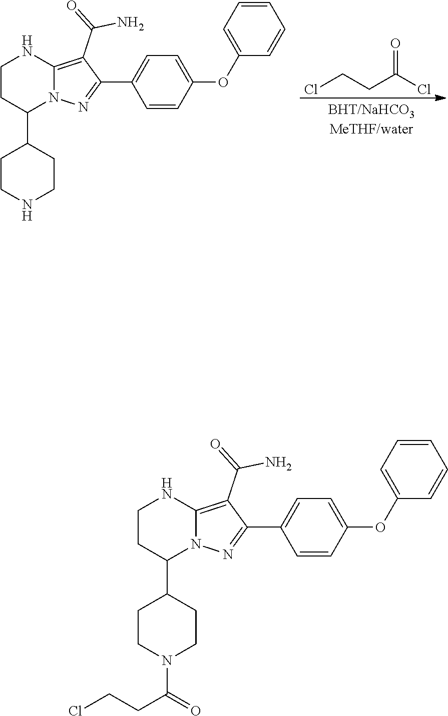 [SMILES: NC(=O)c1c(-c2ccc(Oc3ccccc3)cc2)nn2c1NCCC2C1CCNCC1 ; O=C(Cl)CCCl ; NC(=O)c1c(-c2ccc(Oc3ccccc3)cc2)nn2c1NCCC2C1CCN(C(=O)CCCl)CC1]

2-(4-Phenoxyphenyl)-7-(piperidin-4-yl)-4,5,6,7-tetrahydropyrazolo[1,5-c]pyrimidine-3-carboxamide, racemic, 5 g, 11.976 mmol) was charged into the 250-mL reactor together with 48 mL of 2-methyltetrahydrofuran (9.6 volumes) and 10 mg of butylated hydroxytoluene (0.042 mmol, 0.35 mol %). The suspension was stirred at 10° C. for 20 minutes and cooled to 5-10° C. 43 mL of 7% aq. soln. of NaHCO₃ (59.880 mmol, 3 molEq) was added drop wise in 15 minutes followed by addition of 1.21 mL of 3-chloropropionyl chloride (12.575 mmol, 1.05 molEq) solution in 2.5 mL of 2-methyltetrahydrofurane (0.5 volumes) during 1 hour at 5-10° C. The resulting reaction mixture was stirred at 10° C. for 1 hour, followed by separation of layers. Organic layer was diluted with 50 mL of 2-methyltetrahydrofurane (10 volumes) and washed with 11 mL of 7% NaHCO₃ (9.581 mmol, 0.8 molEq), dried over MgSO₄ and concentrated in vacuo. The remaining foam was dissolved in 25 mL of 2-methyltetrahydrofurane (5 volumes) and 5 mL of n-heptane (1 volume) and stirred at 20-25° C. for 20 hours. The resulting suspension was filtered off and crystal were washed with 5 mL MeTHF/heptane 1:1 mixture (1 volume) and dried for 10 hours at 50° C. and 10 mbar to obtain 4.8 g (78%) of 7-(1-(3-chloropropanoyl)piperidin-4-yl)-2-(4- phenoxyphenyl)-4,5,6,7-tetrahydropyrazolo[1,5-a]pyrimidine-3-carboxamide with 96.63% purity.

### Example 7B

### Synthesis of 7-(143-chloropropanoyl)piperidin-4-yl)-244-phenoxyphenyl)-4,5,6,7-tetrahydropyrazolo[1,5-a]pyrimidine-3-carboxamide racemic

2-(4-Phenoxyphenyl)-7-(piperidin-4-yl)-4,5,6,7-tetrahydropyrazolo[1,5-c]pyrimidine-3-carboxamide base dehydrate racemic, 15 g, 0.036 mol) was charged in a three necked 500 mL flask and suspended in 120 mL (8 volumes) of 2-methyltetrahydrofuran. The white suspension was cooled to 0-5° C. and 129 mL (0.108 mol; 3.27 molEq) of 7% aqueous NaHCO₃ was added dropwise in 30 minutes with temperature not exceeding 5° C. A solution of 3.64 mL (0.038 mol; 1.15 molEq) of 3-chloropropionyl chloride in 30 mL (2 volumes) of 2-methyltetrahydrofuran was added dropwise in 20 minutes with temperature not exceeding 5° C. The mixture was stirred at 0-5° C. until 2-(4-phenoxyphenyl)-7-(piperidin-4-yl)-4,5,6,7-tetrahydropyrazolo[1,5 -a] pyrimidine-3-carboxamide area % by HPLC was below 0.5%. 150 mL (10 volumes) of 2-methyltetrahydrofuran and 45 mL (3 volumes) of water were added and the layers were separated. The water layer was extracted with 90 mL (6 volumes) of 2-methyltetrahydrofuran and the combined organic layers were washed with 45 mL (3 volumes) of 7% aqueous NaHCO3. The layers were separated and the organic layer was dried on sodium sulfate for 20 minutes, inorganics waste was filtered off and the dried organic layer was evaporated to dryness. The obtained white foam was dissolved in 75 mL (5 volumes) of 2-methyltetrahydrofuran and heated to 70° C. Crystallization of thick white suspension occurred during heating and the suspension was stirred for 1 h at 70° C. and then cooled to ambient temperature for stirring overnight. White crystals of racemic 7-(1-(3-chloropropanoyl)piperidin-4-yl)-2-(4-phenoxyphenyl)-4,5,6,7- tetrahydropyrazolo[1,5-a]pyrimidine-3-carboxamide were filtered off and washed two times with 30 mL (2 volumes) of 2- methyltetrahydrofuran /n-heptane 1/1 mixture and dried for 10 h at 50° C. and 10 mbar to obtain 7-(1-(3-chloropropanoyl)piperidin-4-yl)-2-(4-phenoxyphenyl)-4,5,6,7-tetrahydro-pyrazolo[1,5-a]pyrimidine-3-carboxamide in 95% yield and 99.47% purity.

### Example 7C

### Synthesis of (,S′)-7-(1-(3-chloropropanoyl)piperidin-4-yl)-2-(4-phenoxyphenyl)-4,5,6,7-tetrahydropyrazolo11,5-alpyrimidine-3-carboxamide

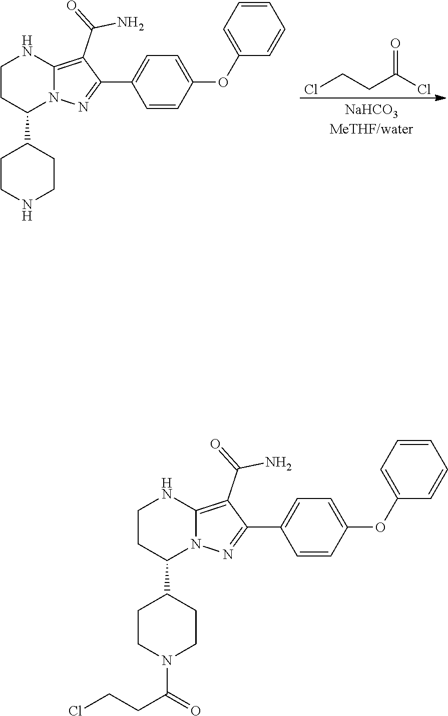 [SMILES: NC(=O)c1c(-c2ccc(Oc3ccccc3)cc2)nn2c1NCC[C@H]2C1CCNCC1 ; O=C(Cl)CCCl ; NC(=O)c1c(-c2ccc(Oc3ccccc3)cc2)nn2c1NCC[C@H]2C1CCN(C(=O)CCCl)CC1]

(S)-2-(4-Phenoxyphenyl)-7-(piperidin-4-yl)-4,5,6,7-tetrahydropyrazolo[1,5-c]pyrimidine-3-carboxamide (3.5 g, 8.380 mmol) was charged into the 100-mL 3-necked flask together with 28 mL of 2-methyltetrahydrofuran (8 volumes). The white suspension was cooled to 5-10° C. and 30.2 mL of 7% aq. soln. of NaHCO₃ (0.025 mol, 3.27 molEq) was added drop wise in 20 minutes followed by addition of 0.96 mL of 3-chloropropionyl chloride (0.010 mol, 1.30 molEq) solution in 7 mL of 2-methyltetrahydrofurane (2 volumes) during 15 minutes at 0-5° C. The resulting reaction mixture was stirred at 10° C. for 0.5 hour, followed by addition of 35 mL (10 volumes) of 2-methyltetrahydrofuran, 10.5 mL (3 volumes) of water and separation of layers. The aqueous layer was extracted with 21 mL (6 volumes) of 2-methyltetrahydrofuran and the combined organic layers were washed with 10,5 ml (3 V) of 7% aq. soln. of NaHCO₃. The layers were separated and the organic layer was dried on sodium sulfate for 20 minutes, inorganics are filtered and the dried organic layer was evaporated to obtain a white foam of (S)-7-(1-(3-chloropropanoyl)piperidin-4-yl)-2-(4-phenoxyphenyl)-4,5,6,7-tetrahydropyrazolo[1,5-a]pyrimidine-3-carboxamide in 99% yield and 95-98% chromatographic purity.

### Example 8A

### Synthesis of 7-(1-acryloylpiperidin-4-yl)-2-(4-phenoxyphenyl)-4,5,6,7-tetrahydropyrazolo[1,5-a]pyrimidine-3-carboxamide racemic from 7-(1-(3-chloropropanoyl)piperidin-4-yl)-2-(4-phenoxyphenyl)-4,5,6,7-tetrahydropyrazolo[1,5-a]pyrimidine-3-carboxamide racemic

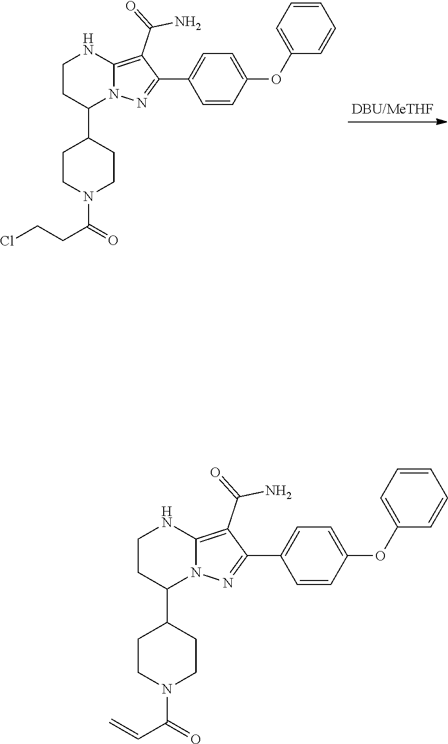 [SMILES: NC(=O)c1c(-c2ccc(Oc3ccccc3)cc2)nn2c1NCCC2C1CCN(C(=O)CCCl)CC1 ; C=CC(=O)N1CCC(C2CCNc3c(C(N)=O)c(-c4ccc(Oc5ccccc5)cc4)nn32)CC1]

7-(1-(3-Chloropropanoyl)piperidin-4-yl)-2-(4-phenoxyphenyl)-4,5,6,7-tetrahydropyrazolo [1,5-a]pyrimidine-3-carboxamide (1 g, 1.968 mmol) was suspended in 12 mL of anhydrous 2-methyltetrahydrofuran (12 volumes) under argon. 736 μL of 1,8-diazabicyclo[5.4.0]undec-7-ene (DBU, 4.921 mmol, 2.5 molEq) was added drop wise at 20-25° C. during 1 hour. Reaction mixture (white suspension) was stirred at 20-25° C. for 20 h resulting with 52% conversion to 7-(1-acryloylpiperidin-4-yl)-2-(4-phenoxyphenyl)-4,5,6,7-tetrahydropyrazolo[1,5 -a] pyrimidine-3-carboxamide racemic. The mixture was then warmed up and stirred 10 h at 40° C. and additional 4 hours at 50° C. resulting with 90% conversion. Reaction mixture was then cooled to 20-25° C., diluted with 100 mL of 2-methyltetrahydrofurane and 100 mL of water and pH was adjusted from >14 to 6.5 with 1M HC_{1.} Layers were separated and organic layer was evaporated yielding 1 g of crude 7-(1-acryloylpiperidin-4-yl)-2-(4-phenoxyphenyl)-4,5,6,7-tetrahydropyrazolo[1,5-a] pyrimidine-3-carboxamide. The remaining solid was crystallized from 6 mL of ethyl acetate (6 volumes) and filtered off at 0-5° C. yielding 610 mg (54%) of racemic 7-(1-acryloylpiperidin-4-yl)-2-(4-phenoxyphenyl)-4,5,6,7-tetrahydropyrazolo[1,5 -a] pyrimidine-3-carboxamide_as a white powdery solid.

### Example 8B

### Synthesis of 7-(1-acryloylpiperidin-4-yl)-244-phenoxyphenyl)-4,5,6,7-tetrahydropyrazolo[1,5-a]pyrimidine-3-carboxamide racemic from 7-(1-(3-chloropropanoynpiperidin-4-yl)-2-(4-phenoxyphenyl)-4,5,6,7-tetrahydropyrazolo[1,5-a]pyrimidine-3-carboxamide racemic

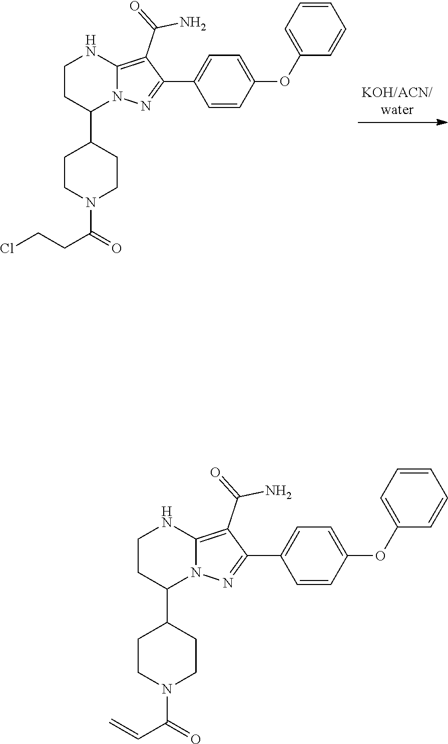 [SMILES: NC(=O)c1c(-c2ccc(Oc3ccccc3)cc2)nn2c1NCCC2C1CCN(C(=O)CCCl)CC1 ; C=CC(=O)N1CCC(C2CCNc3c(C(N)=O)c(-c4ccc(Oc5ccccc5)cc4)nn32)CC1]

7-(1-(3-Chloropropanoyl)piperidin-4-yl)-2-(4-phenoxyphenyl)-4,5,6,7-tetrahydropyrazolo [1,5-a]pyrimidine-3-carboxamide 7 g, 13.655 mmol) was suspended in 35 mL of acetonitrile (5 volumes) and 35 mL of water (5 volumes). 992 mg of KOH (powder, 85%, 15.021 mmol, 1.10 molEq) was added at 20-25° C. in one portion. The mixture was warmed up and stirred 3 h at 55-60° C. resulting with yellowish 2-layer solution with complete conversion. Reaction mixture was then treated with active charcoal for 30 minutes, filtered off and the filtrate was cooled to 20-25° C. and stirred for 20 hours resulting with thick white suspension. The solid was filtered off, washed with 20 mL of acetonitrile/water 1:1 mixture (2.9 volumes) and dried at 10 mbar and 55° C. for 10 hours yielding 3.46 g (54%) of crude 7-(1-acryloylpiperidin-4-yl)-2-(4-phenoxyphenyl)-4,5,6,7-tetrahydropyrazolo[1,5-a] pyrimidine-3-carboxamide with 98.16% purity.

Mother liquor was concentrated in vacuo, and in the remaining mixture in water dichloromethane (50 mL) was added. Layers were separated and aqueous layer was extracted two more times with dichloromethane. Combined organic layers were evaporated to the foamy residue which was then dissolved in acetonitrile/water 1:1 mixture (30 mL), seeded with 7-(1-acryloylpiperidin-4-yl)-2-(4-phenoxyphenyl)-4,5,6,7-tetrahydropyrazolo[1,5-a]pyrimidine-3-carboxamide racemic and stirred overnight at 20-25° C. The obtained suspension was filtered off and solid was washed with acetonitrile/water 1:1 mixture (5 mL) and dried at 10 mbar and 55° C. for 10 hours yielding additional 1.03 g of 741-acryloylpiperidin-4-yl)-2-(4-phenoxyphenyl)-4,5,6,7-tetrahydropyrazolo[1,5-a]pyrimidine-3-carboxamide racemic with 97.63% purity (total yield 70%).

### Example 8C

### Synthesis of 01-7-(1-acryloylpiperidin-4-yl)-2-(4-phenoxyphenyl)-4,5,6,7-tetrahydropyrazolo[1,5-a]pyrimidine-3-carboxamide from (S)-7-(1-(3-chloropropanoyl)piperidin-4-yl)-2-(4-phenoxyphenyl)-4,5,6,7-tetrahydropyrazolo[1,5- a]pyrimidine-3-carboxamide

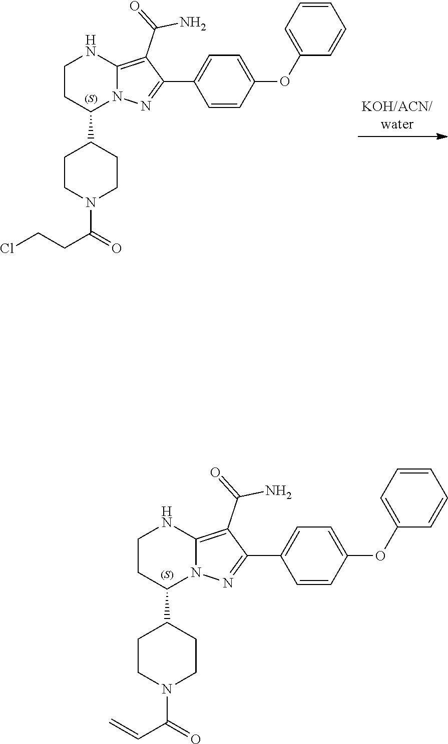

(S)-7-(1-(3-Chloropropanoyl)piperidin-4-yl)-2-(4-phenoxyphenyl)-4,5,6,7-tetrahydro-pyrazolo [1,5-a]pyrimidine-3-carboxamide (3.22 g, 6.334 mmol) was dissolved in 16 mL of acetonitrile (5 volumes) and 16 mL of water (5 volumes). 373 mg of KOH (powder, 85%, 15.021 mmol, 1.05 molEq) was added at 20-25° C. in one portion. The mixture was warmed up and stirred for 4 h at 45-50° C. when additional amount of KOH was added (22 mg, total 1.10 molEq) and the mixture was continued stirring at 50° C. for 1 more hour. The temperature was then lowered to 20-25° C. and the obtained mixture was stirred overnight. Acetonitrile was evaporated out and the remaining mixture in water was extracted with dichloromethane (3×50 mL). Combined organic layers were evaporated to the foamy residue. The foamy residue may be dissolved in tetrahydrofuran (3.5 volumes) and the solution added drop wise into a pre-cooled methyl-tert-butyl ether (30 volumes) at 0 C during 20 minutes. The resulting suspension may be stirred at 0 C for another 2 hours, filtered off in nitrogen stream. The solid may be washed with cold methyl-tert-butyl ether and dried on Buchner funnel in a nitrogen stream for 16 hours yielding Zanubrutinib.

Further aspects and embodiments of the disclosure are set out in the numbered clauses below:

1. A process for preparing Zanubrutinib or a salt thereof, comprising a step of reacting a compound (IX) with compound (VIII) to obtain compound (VII):

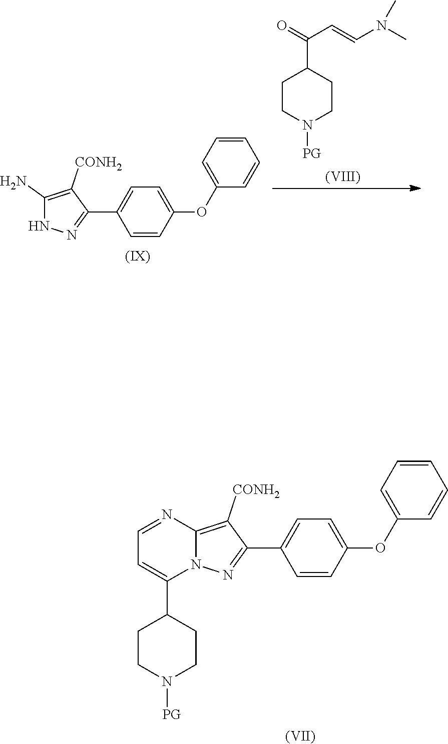

wherein PG is a protecting group; and converting the compound of formula (VII) to Zanubrutinib or a salt thereof.
2. A process according to Clause 1 wherein PG is selected from acetyl, benzyl, methyl, benzoyl, toluoyl, methoxycarbonyl, ethoxycarbonyl, benzyloxycarbonyl, tert-butyloxycarbonyl, allyloxycarbonyl, 4-methoxybenzyl, para-methoxybenzylcarbonyl, 3,4-dimethoxybenzyoyl, propionyl, butyryl, phenylacetyl, phenoxyacetyl, trityl, 2,2,2-trichloroethoxycarbonyl, carbobenzoxy, 4-methoxybenzyloxycarbonyl, 9-fluorenylmethoxycarbonyl, 2-iodoethoxycarbonyl, 4-methoxy-2,3,6-trimethylbenzenesulfonyl, methanesulfonyl, para-toluenesulfonyl, phenyl sulfonyl, trifluorocarbonyl, 2-trimethylsilylethoxycarbonyl, and 4-nitrobenzenesulfonyl.
3. A process according to any of Clauses 1 or 2, wherein PG is, methoxycarbonyl, ethoxycarbonyl, benzyloxycarbonyl, tert-butyloxycarbonyl, para-methoxybenzylcarbonyl, 3,4-dimethoxybenzyoyl, phenylacetyl, phenoxyacetyl, or 4-methoxybenzyloxycarbonyl.
4. A process according to any of Clauses 1, 2, or 3, wherein PG is tert-butyloxycarbonyl.
5. A process according to any of Clauses 1, 2, 3, or 4, wherein the reaction is carried out in the presence of an acid, optionally an organic acid, wherein the acid is optionally selected from the group consisting of acetic acid, trifluoroacetic acid.
6. A process according to any of Clauses 1, 2, 3, 4, or 5, wherein the reaction is carried out in a solvent, optionally an aprotic solvent.
7. A process according to any of Clauses 1, 2, 3, 4, 5, or 6, wherein the reaction is carried out in a solvent selected from an aromatic hydrocarbon, an aromatic alcohol, a polar aprotic solvent and a polar protic solvent, optionally wherein the solvent is toluene, mesitylene, chlorobenzene, benzonitrile, benzyl alcohol, xylene, N,N-dimethylformamide (DMF), acetonitrile (ACN), methylethylketone (MEK), tetrahydrofuran (THF), dimethylacetate (DMAc), dimethylsulfoxide (DMSO), N-methylpyrrolidone (NMP), acetone, methylisobutylketone (MIBK), dichloromethane (DCM), methanol, ethanol, 1-propanol, 2-propanol, 1-butanol.
8. A process according to any of Clauses 1, 2, 3, 4, 5, 6, or 7, wherein the reaction is carried out at a temperature of: about 50° C. to about 130° C., about 70° C. to about 120° C., about 80° C. to about 110° C., about 90° C. to about 100° C., or about 95° C.
9. A process according to Clause 8, wherein the reaction mixture is heated for: about 6 to 24 hours, about 8 to about 20 hours, or about 10 to about 14 hours, or about 12 hours.
10. A process according to any of Clauses 8 or 9, wherein the reaction mixture is subsequently cooled to: about 10° C. to about 40° C., about 15° C. to about 30° C. or about 20° C. to about 25° C., and optionally maintained for about 5 to about 20 hours, about 8 to about 15 hours, or about 10 hours.
11. A process according to any of Clauses 1, 2, 3, 4, 5, 6, 7, 8, 9, or 10, wherein the compound of formula (VII) is isolated by filtration.
12. A process according to any of Clauses 1, 2, 3, 4, 5, 6, 7, 8, 9, 10, or 11, further comprising, sequentially in any order or simultaneously, the steps of reducing and deprotecting the compound (VII), to obtain compound (VI):

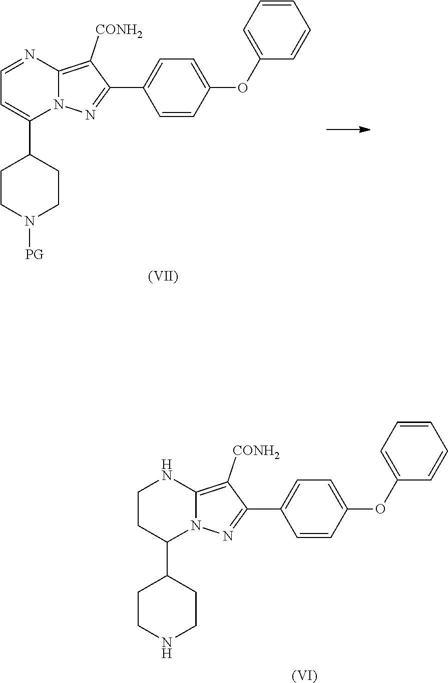

13. A process according to Clause 12, comprising reducing the compound (VII) to form a compound of formula (VII-A):

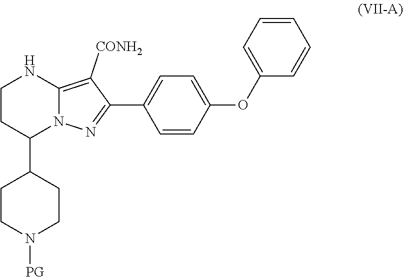

and deprotecting the compound (VI-A) to form the compound (VI), optionally wherein the deprotection is carried out without isolation of the compound of formula (VII-A).
14. A process according to Clause 12, comprising deprotecting the compound (VII) to form a compound (VII-B):

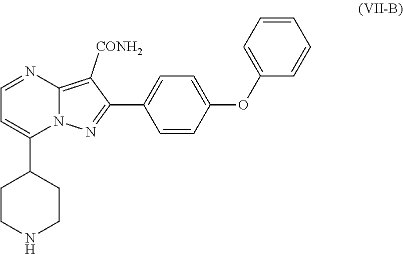 [SMILES: NC(=O)c1c(-c2ccc(Oc3ccccc3)cc2)nn2c(C3CCNCC3)ccnc12]

and reducing the compound (VII-B) to form the compound (VI), optionally wherein the reduction is carried out without isolation of the compound of formula (VII-B).
15. A process according to Clause 12, comprising simultaneously reducing and deprotecting the compound (VII) to form the compound (VI).
16. A process according to any of Clauses 12, 13, 14, and 15, wherein the reduction is carried out by hydrogenation in the presence of hydrogen gas and a catalyst, optionally where the catalyst comprises palladium or platinum, or wherein the reduction is carried out with a reducing agent, optionally sodium borohydride or sodium cyanoborohydride.
17. A process according to Clause 16, wherein the hydrogenation is carried out in the presence of an acid, optionally an organic acid, optionally acetic acid; or optionally wherein the acid is malic acid, mandelic acid, camphorsulfonic acid, tartaric acid, dibenzoyltartaric acid, di-p-toluoyltartaric acid, or optically active isomers thereof; optionally wherein the acid is L-malic acid, L-mandelic acid, L-camphorsulfonic acid, L-tartaric acid, dibenzoyl-L-tartaric acid, di-p-toluoyl-L-tartaric acid, D-malic acid, D-mandelic acid, D-camphorsulfonic acid, D-tartaric acid, dibenzoyl-D-tartaric acid, di-p-toluoyl-D-tartaric acid.
18. A process according to any of Clauses 16 or 17, wherein the hydrogenation is carried out in an aprotic solvent, optionally THF, DMSO, or dioxane, or mixtures thereof, and preferably wherein the solvent is THF.
19. A process according to any of Clauses 16, 17 or 18, wherein the hydrogenation is carried out under a pressure of: about 1 bar to about 10 bar, about 2 bar to about 9 bar, about 3 bar to about 8 bar, about 4 bar to about 7 bar, about 5 to about 6 bar.

20. A process according to any of Clauses 16, 17, 18, or 19, wherein the hydrogenation is carried out at a temperature of about 20° C. to about 100° C., about 30° C. to about 90° C., about 40° C. to about 80° C., about 50° C. to about 70° C., or about 60° C.

21. A process according to any of Clauses 16, 17, 18, 19, or 20, wherein the hydrogenation is carried out over a period of about 10 hours to about 48 hours, about 14 hours to about 40 hours, about 18 hours to about 36 hours, about 20 hours to about 30 hours, or about 24 hours.
22. A process according to any of Clauses 12, 13, 14, 15, 16, 17, 18, 19, 20, or 21, wherein the deprotection is carried out in the presence of an acid or a base, or wherein the deprotection is effected during the reduction step.
23. A process according to any of Clauses 1, 2, 3, 4, 5, 6, 7, 8, 9, 10, 11, 12, 13, 14, 15, 16, 17, 18, 19, 20, 21, or 22, wherein the compound of formula (IX) is obtained by basification of an acid addition salt of compound of formula (X):

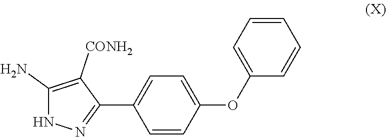 [SMILES: NC(=O)c1c(-c2ccc(Oc3ccccc3)cc2)n[nH]c1N]

wherein the salt is with methane sulfonic acid, p-toluene sulfonic acid or polyphosphoric acid.
24. A process according to Clause 23, wherein the compound of formula (X) is prepared by hydrolysis of a compound of formula (XI) using methanesulfonic acid:

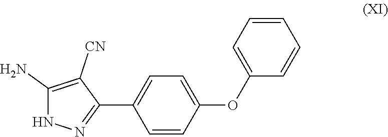 [SMILES: N#Cc1c(-c2ccc(Oc3ccccc3)cc2)n[nH]c1N]

followed by basification.
25. A process according to any of Clauses 1, 2, 3, 4, 5, 6, 7, 8, 9, 10, or 11, wherein the compound (VII) is converted to Zanubrutinib by a process comprising:
(a) subjecting the compound (VII) to reduction and deprotection sequentially in any order, or simultaneously, to form a compound (VI):

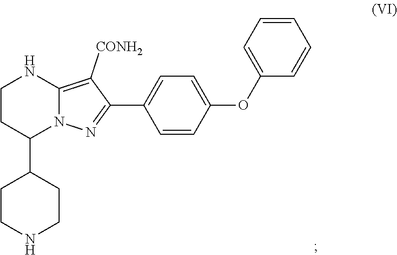 [SMILES: NC(=O)c1c(-c2ccc(Oc3ccccc3)cc2)nn2c1NCCC2C1CCNCC1]

b) chiral resolution of the compound (VI) to form a compound (III):

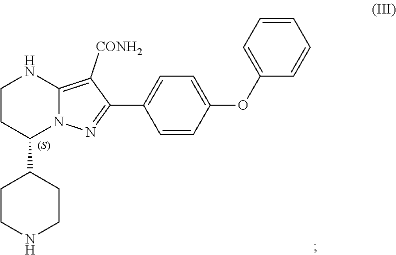

and
c) N-substitution of the compound (III) to form Zanubrutinib (I):

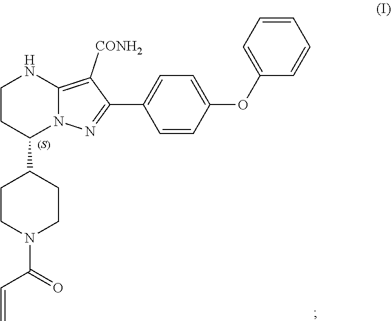

26. A process according to Clause 25 wherein step (a) is carried out according to the process of any of Clauses 12, 13, 14, 15, 16, 17, 18, 19, 20, 21, 22, 23, or 24.
27. A process according to any of Clauses 25 or 26, wherein step (b) is carried out by diastereomeric crystallization using a chiral acid, optionally wherein the chiral acid is selected from the group consisting of: malic acid, mandelic acid, camphorsulfonic acid, tartaric acid, dibenzoyltartaric acid, di-p-toluoyltartaric acid, and optionally wherein the chiral acid is selected from the group consisting of: dibenzoyl-L-tartaric acid, dibenzoyl-D-tartaric acid, di-p-toluoyl-D-tartaric acid, di-p-toluoyl-L-tartaric acid, L-malic acid, D-malic acid, L-tartaric acid, D-tartaric acid, L-mandelic acid, D-mandelic acid, L-camphorsulfonic acid, or D-camphorsulfonic acid.
28. A process according to any of Clauses 25, 26, or 27, wherein step (b) is carried out by diastereomeric crystallization using dibenzoyl-L-tartaric acid or L-malic acid, optionally L-malic acid.
29. A process according to Clause 27 or 28, wherein the diastereomeric crystallisation is carried out in a polar solvent, optionally a C₁₋₆ alcohol, water or mixtures of one or more of a C₁₋₆ alcohol and water, or wherein the polar solvent is methanol, ethanol, n-propanol, 2-propanol, n-butanol, 2-butanol, THF, acetonitrile, or their mixtures with water, or wherein the polar solvent is: methanol/water, ethanol, ethanol/water, n-propanol/water, 2-propanol/water, n-butanol/water, THF/water, acetonitrile/water.
30. A process according to any of Clauses 27, 28 or 29, wherein the diastereomeric crystallisation is carried out by heating a solution comprising the compound (VI) with the chiral acid in a solvent, to a temperature of about 50° C. to about 100° C., about 60° C. to about 90° C., about 75° C. to about 85° C., or about 80° C. to about 83° C.
31. A process according to any of Clauses 27, 28, 29, or 30, wherein the diastereomeric crystallisation comprises reacting the compound (VI) with a chiral acid, optionally wherein the chiral acid is selected from the group consisting of:: dibenzoyl-L-tartaric acid, dibenzoyl-D-tartaric acid, di-p-toluoyl-D-tartaric acid, di-p-toluoyl-L-tartaric acid, L-malic acid, D-malic acid, L-tartaric acid, D-tartaric acid, L-mandelic acid, D-mandelic acid, L-camphorsulfonic acid, or D-camphorsulfonic acid,
to form the compound (V):
wherein

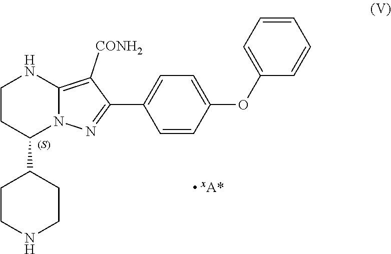

A* is a chiral acid; and
x is 0.5 for dibenzoyltartaric acid, di-p-toluoyl-tartaric acid or tartaric acid; and x is 1 for mandelic acid, malic acid or camphorsulfonic acid;
isolating the compound (V);
basifying the compound (V) to form the compound (III):

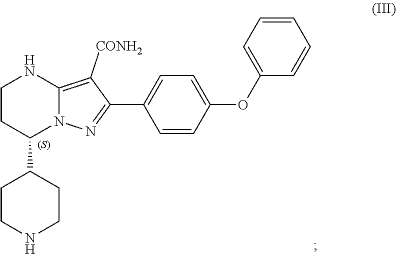

and
optionally isolating the compound (III),
wherein preferably the compound (III) is not isolated.
32. A process according to any of Clauses 25, 26, 27, 28, 29, 30, or 31, wherein steps b) and c) comprise:
chiral resolution of the compound (VI) to form a compound

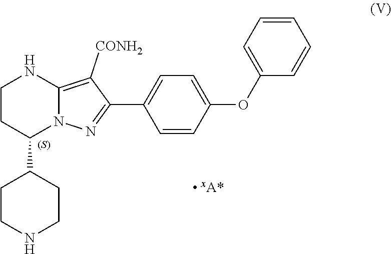

wherein A* is a chiral acid optionally wherein the chiral acid is selected from the group consisting of: malic acid, mandelic acid, camphorsulfonic acid, tartaric acid, dibenzoyltartaric acid, di-p-toluoyltartaric acid, and optionally wherein the chiral acid is selected from the group consisting of: dibenzoyl-L-tartaric acid, dibenzoyl-D-tartaric acid, di-p-toluoyl-D-tartaric acid, di-p-toluoyl-L-tartaric acid, L-malic acid, D-malic acid, L-tartaric acid, D-tartaric acid, L-mandelic acid, D-mandelic acid, L-camphorsulfonic acid, or D-camphorsulfonic acid; and
x is 0.5 for dibenzoyltartaric acid, di-p-toluoyl-tartaric acid or tartaric acid; and x is 1 for mandelic acid, malic acid or camphorsulfonic acid;
basifying the compound (V) to form a reaction mixture comprising the compound (III)

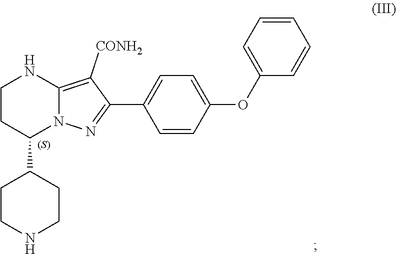

and reacting the compound (III) in situ by N-alkylation to form Zanubrutinib.
33. A process according to any of Clauses 25, 26, 27, 28, 29, 30, 31, or 32, wherein step (c) comprises:
(i) reacting the compound (III) with compound (IV-A):

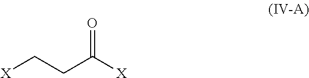

to prepare a compound of formula (II):

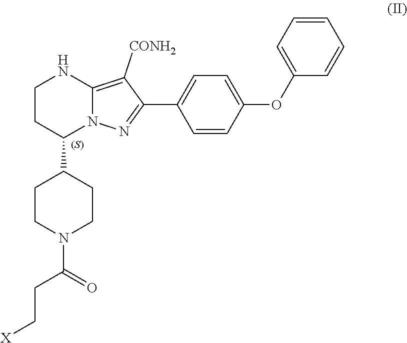

wherein X at either occurrence is the same or different and represents chloro or bromo, optionally wherein X at both occurrences is chloro; and
   (ii) elimination to form Zanubrutinib (I).
   34. A process according to Clause 32, wherein:
the compound (III) is obtained by the process of claim 31, preferably wherein the compound (III) is not isolated; and/or
the compound of formula (II) is used from the reaction mixture without isolating and/or without further purification.
   35. A process according to Clause 33 or Clause 34, wherein step (i) is carried out in a polar solvent, optionally wherein the solvent comprises acetonitrile or a C₁₋₆ alcohol, or a mixture of acetonitrile and water, or a mixture of a C₁₋₆ alcohol and water, or wherein the solvent is a mixture of acetonitrile and water, or a mixture of n-propanol and water.
   36. A process according to any of Clauses 33, 34, or 35, wherein step (i) is carried out in the presence of a base.
   37. A process according to Clause 36, wherein the base is selected from an alkali metal carbonate, an alkali metal bicarbonate, or a tertiary C₁-4 alkyl amine; and optionally sodium hydrogen carbonate.
   38. A process according to Clause 35, 36 or 37, wherein the solvent is selected from the group consisting of acetonitrile, tetrahydrofuran, 2-methyl-tetrahydrofuran, methylethylketone, dichloromethane, ethyl acetate, toluene, acetone, Ci to C₆ alcohol, optionally wherein the solvent is in a mixture with water, or wherein the solvent is a mixture of acetonitrile and water or a mixture of n-propanol and water.
   39. A process according to any of Clauses 33, 34, 35, 36, 37, or 38, wherein step (i) is carried out at a temperature of about −10° C. to about 20° C., about −5° C. to about 15° C., about 0° C. to about 10° C., or about 5° C. to about 10° C.
   40. A process according to any of Clauses33, 34, 35, 36, 37, 38, or 39, wherein step (ii) comprises reacting compound (II) with a base in a solvent.
   41. A process according to Clause 40, wherein the base is selected from: potassium hydroxide, sodium hydroxide, 1,8-Diazabicyclo[5.4.0]undec-7-ene (DBU), t-BuOK, NaOMe, NaOEt, and optionally potassium hydroxide.
   42. A process according to Clause 40 or Clause 41, wherein the solvent is selected from the group consisting of 2-methyltetrahydrofuran (MeTHF), THF, ethyl acetate (EtOAc), acetonitrile, C₁-C₄ alcohol optionally methanol, ethanol, n-propanol, 2-propanol, t-butanol, or mixtures thereof with water; and optionally wherein the solvent is selected from a mixture of acetonitrile and water, or n-propanol/water.
   43. A process according to any of Clauses 40, 41 or 42, wherein the reaction is carried out at a temperature of about 10° C. to about 70° C., about 15° C. to about 65° C., about 20° C. to about 60° C., about 30° C. to about 55° C., about 40-50° C., or about 45-50° C.
   44. A process according to any of Clauses 25, 26, 27, 28, 29, 30, 31, or 32, wherein step (c) comprises reacting the compound (III) with a compound (IV-B):

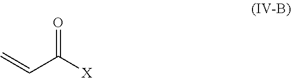

wherein X is chloro or bromo, optionally chloro.
   45. A process according to Clause 44, wherein the reaction is carried out in the presence of in a polar aprotic solvent, in the presence of a base.
   46. A process according to Clause 45, wherein the base is selected from: potassium hydroxide, sodium hydroxide, 1,8-diazabicyclo[5.4.0]undec-7-ene.
   47 A process according to any of Clauses 45 or 46, wherein the polar aprotic solvent is selected from the group consisting of acetonitrile, tetrahydrofuran, 2-methyl-tetrahydrofuran, water, or mixtures thereof, optionally wherein the solvent is 2-methyl-tetrahydrofuran.
   48. A process according to any of Clauses 44, 45, 46, or 47, wherein the reaction is carried out at a temperature of about 10° C. to about 40° C., about 15° C. to about 30° C., about 20° C. to about 25° C.
   49. A process for preparing Zanubrutinib comprising the steps of:
(A) reacting a compound (IX) with a compound (VIII) according to any of Clauses 1, 2, 3, 4, 5, 6, 7, 8, 9, 10, or 11, to form the compound (VII):

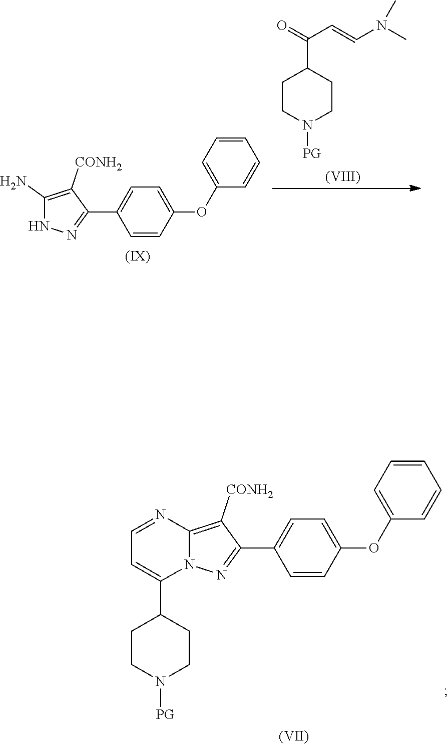

(B) reducing and deprotecting the compound (VII) according to any of Clauses 12, 13, 14, 15, 16, 17, 18, 19, 20, 21, or 22 to form a compound (VI):

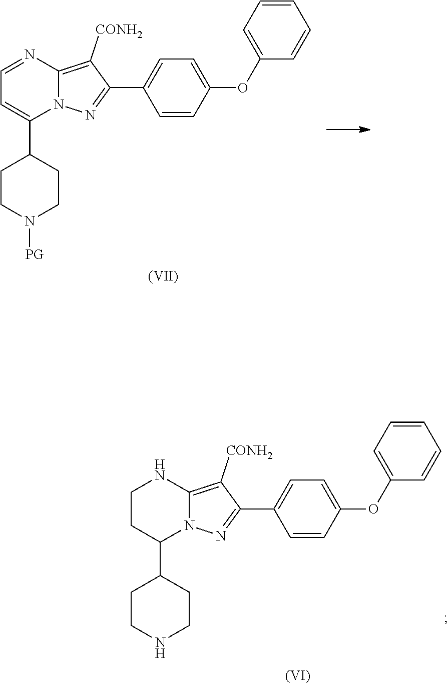

(C) chiral resolution of the compound (VI) according to any of Clauses 27, 28, 29, or 30, or 31, to form a compound (III):

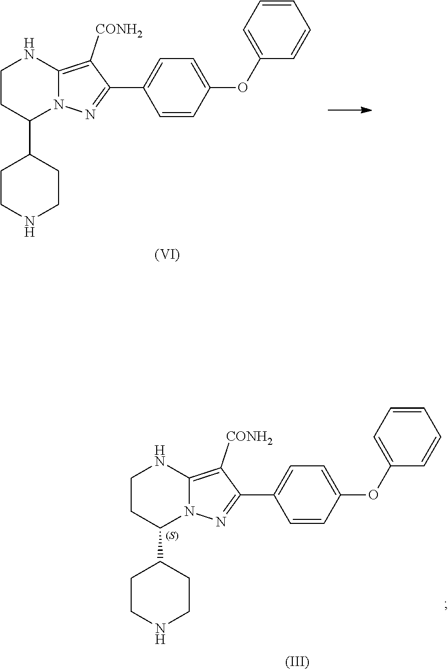

and
(D) N-substitution of the compound (III) according to any of Clauses 33, 34, 35, 36, 37, 38, 39, 40, 41, 42, 43, 44, 45, 46, 47, or 48:

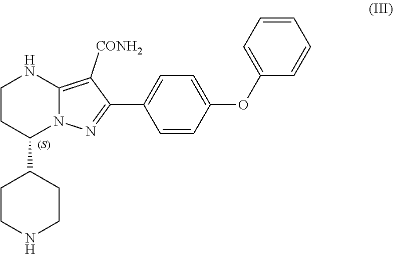

to form Zanubrutinib.
   50. A process according to Clause 49, wherein steps (C) and (D) comprise:
reacting the compound (VI) with a chiral acid, optionally wherein the chiral acid is selected from the group consisting of: malic acid, mandelic acid, camphorsulfonic acid, tartaric acid, dibenzoyltartaric acid, di-p-toluoyltartaric acid, and optionally wherein the chiral acid is selected from the group consisting of: dibenzoyl-L-tartaric acid, dibenzoyl-D-tartaric acid, di-p-toluoyl-D-tartaric acid, di-p-toluoyl-L-tartaric acid, L-malic acid, D-malic acid, L-tartaric acid, D-tartaric acid, L-mandelic acid, D-mandelic acid, L-camphorsulfonic acid, or D-camphorsulfonic acid,
to form the compound (V):

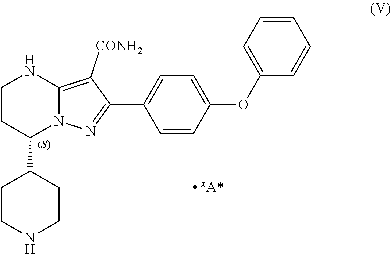

wherein
   A* is a chiral acid; and x is 0.5
   x is 0.5 for dibenzoyl-D-tartaric acid, dibenzoyl-L-tartaric acid, di-p-toluoyl-D-tartaric acid, di-p-toluoyl-L-tartaric, D-tartaric acid, or L-tartaric acid; and x is 1 for D-mandelic, L-mandelic acid, D-malic acid, L-malic acid, D-camphorsulfonic acid, or L-camphorsulfonic acid;
isolating the compound (V);
basifying the compound (V) to form the compound (III):

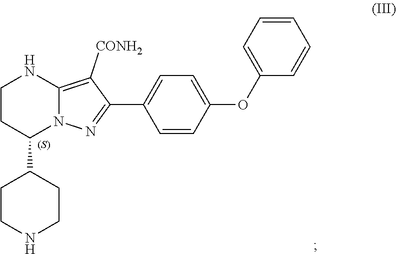

reacting the compound (III) by N-alkylation according to any of Clauses 33, 34, 35, 36, 37, 38, 39, 40, 41, 42, 43, 44, 45, 46, or 47, or 48, to form Zanubrutinib, wherein the compound (III) is not isolated after the basifying step.
   51. A process according to Clause 49 or Clause 50, wherein step (D) is carried out according to any of Clauses 33, 34, 35, 36, 37, 38, 39, 40, 41, 42, 43, 44, 45, 46, 47, or 48.
   52. A process according to Clause 49, 50, or 51, wherein the compound of formula (IX) is prepared according to any of Clauses 24 or 25.
   53. A process according to any of the preceding clauses, further comprising the step of converting Zanubrutinib into a salt thereof.
   54. A compound selected from:

### Formula (VII):

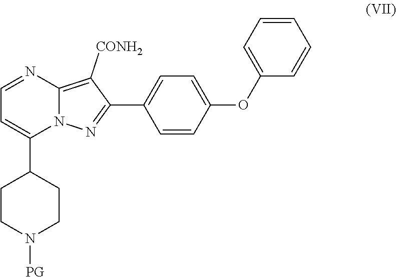

wherein PG is a protecting group, optionally acetyl, benzyl, methyl, benzoyl, toluoyl, methoxycarbonyl, ethoxycarbonyl, benzyloxycarbonyl, tert-butyloxycarbonyl, allyloxycarbonyl, 4-methoxybenzyl, para-methoxybenzylcarbonyl, 3,4-dimethoxybenzyoyl, propionyl, butyryl, phenylacetyl, phenoxyacetyl, trityl, 2,2,2-trichloroethoxycarbonyl, carbobenzoxy, 4-methoxybenzyloxycarbonyl, 9-fluorenylmethoxycarbonyl, 2-iodoethoxycarbonyl, 4-methoxy-2,3,6-trimethylbenzenesulfonyl, methanesulfonyl, para-toluenesulfonyl, phenyl sulfonyl, trifluorocarbonyl, 2-trimethyl silylethoxycarbonyl, 4-nitrobenzenesulfonyl; or

### Formula (II):

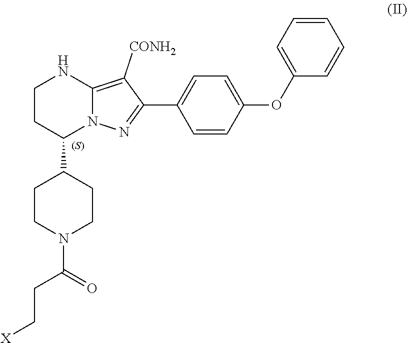

wherein X is chloro or bromo.
55. A compound of formula (VII) according to Clause 52:

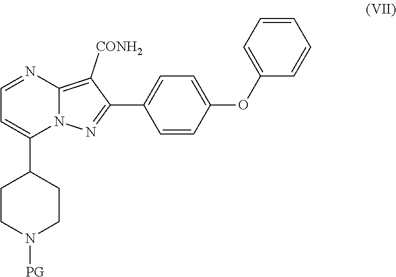

wherein PG is methoxycarbonyl, ethoxycarbonyl, benzyloxycarbonyl, or tert-butyloxycarbonyl, and optionally wherein PG is tert-butyloxycarbonyl.
56. A compound of formula (VII) according to Clause 55 which is:

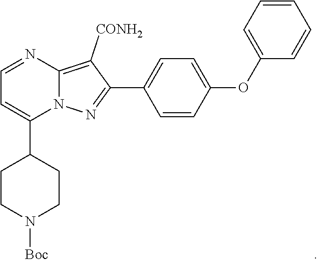 [SMILES: CC(C)(C)OC(=O)N1CCC(c2ccnc3c(C(N)=O)c(-c4ccc(Oc5ccccc5)cc4)nn23)CC1]

57. A compound according to Clause 56, preferably in crystalline form, and more preferably which is characterized by an X-ray powder diffraction pattern having peaks at 7.2, 9.4, 10.0, 12.6 and 16.6 degrees 2-theta±0.2 degrees 2-theta; or an X-ray powder diffraction pattern substantially as depicted in FIG. 5.
58. A compound of formula (II) according to Clause 54 which is:

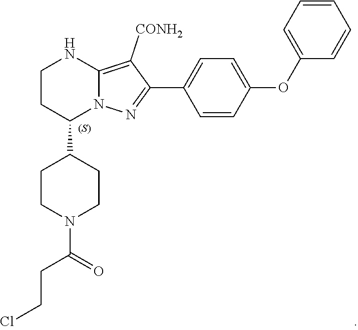

59. Compound (X) which is the methanesulfonate salt, having the formula:

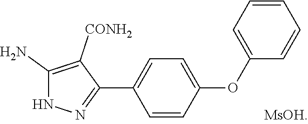 [SMILES: NC(=O)c1c(-c2ccc(Oc3ccccc3)cc2)n[nH]c1N]

60. Compound (X) according to Clause 59, which is crystalline, optionally characterized by an X-ray powder diffraction pattern having peaks at: 5.2, 10.5, 17.0, 19.0, and 21.0 degrees 2-theta±0.2 degrees 2-theta, and/or an X-ray powder diffraction pattern substantially as depicted in FIG. 1.
61. Compound (X) according to Clause 59, which is crystalline, which is characterized by an X-ray powder diffraction pattern having peaks at: at 5.8, 11.1, 13.7, 17.3 and 18.3 degrees 2-theta±0.2 degrees 2-theta, and/or an X-ray powder diffraction pattern substantially as depicted in FIG. 2.
62. Compound (X) according to Clause 59, which is crystalline, which is characterized by an X-ray powder diffraction pattern having peaks at: at 5.4, 8.4, 9.2, 10.8 and 16.2 degrees 2-theta±0.2 degrees 2-theta, and/or an X-ray powder diffraction pattern substantially as depicted in FIG. 3.
63. Use of a compound according to any of Clauses 54, 55, 56, 57, 58, 59, 60, 61, or 62, for preparing Zanubrutinib or optionally a salt thereof
64. A process according to any of Clauses 1 to 53, further comprising combining the Zanubrutinib or a salt of Zanubrutinib with at least one pharmaceutically acceptable excipient to form a pharmaceutical formulation, optionally wherein the pharmaceutical formulation is an oral dosage form.

1. A process for preparing Zanubrutinib or a salt thereof, comprising a step of reacting a compound (IX) with compound (VIII) to obtain compound (VII): 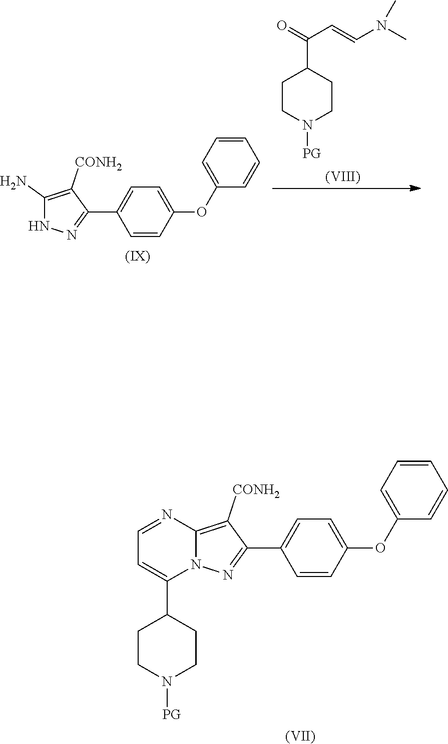
wherein PG is a protecting group; and converting the compound of formula (VII) to Zanubrutinib or a salt thereof.

2. A process according to claim 1 wherein PG is selected from acetyl, benzyl, methyl, benzoyl, toluoyl, methoxycarbonyl, ethoxycarbonyl, benzyloxycarbonyl, tert-butyloxycarbonyl, allyoxycarbonyl, 4-methoxybenzyl, para-methoxybenzylcarbonyl, 3,4-dimethoxybenzyoyl, propionyl, butyryl, phenylacetyl, phenoxyacetyl, trityl, 2,2,2-trichloroethoxycarbonyl, carbobenzoxy, 4-methoxybenzyloxycarbonyl, 9-fluorenyl methoxycarbonyl, 2-iodoethoxycarbonyl, 4-methoxy-2,3,6-trimethylbenzenesulfonyl, methanesulfonyl, para-toluenesulfonyl, phenylsulfonyl, trifluorocarbonyl, 2-trimethylsilylethoxycarbonyl, and 4-nitrobenzenesulfonyl.

3. A process according to claim 1, wherein PG is methoxycarbonyl, ethoxycarbonyl, benzyloxycarbonyl, tert-butyloxycarbonyl, para-methoxybenzylcarbonyl, 3,4-dimethoxybenzyoyl, phenylacetyl, phenoxyacetyl, or 4-methoxybenzyloxycarbonyl.

4. A process according to claim 1, wherein PG is tert-butyloxycarbonyl.

5. A process according to claim 1, wherein the reaction is carried out in the presence of an acid.

6. A process according to claim 1, wherein the reaction is carried out in a solvent.

7. A process according to claim 1, wherein the reaction is carried out in a solvent selected from an aromatic hydrocarbon, an aromatic alcohol, a polar aprotic solvent or a polar protic solvent.

8. A process according to claim 1, wherein the compound (VII) is converted to Zanubrutinib by a process comprising:
(a) subjecting the compound (VII) to reduction and deprotection sequentially in any order, or simultaneously, to form a compound (VI): 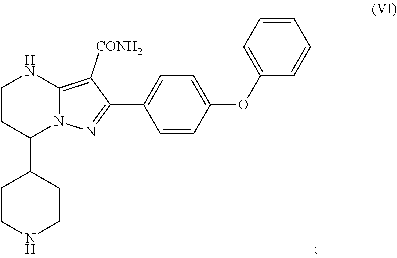 [SMILES: NC(=O)c1c(-c2ccc(Oc3ccccc3)cc2)nn2c1NCCC2C1CCNCC1]
b) chiral resolution of the compound (VI) to form a compound (III): 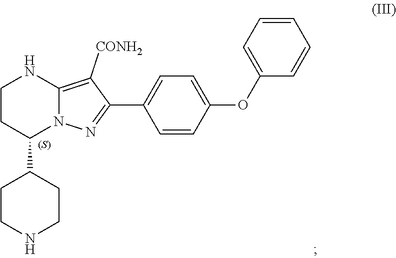
and
c) N-substitution of the compound (III) to form Zanubrutinib (I):

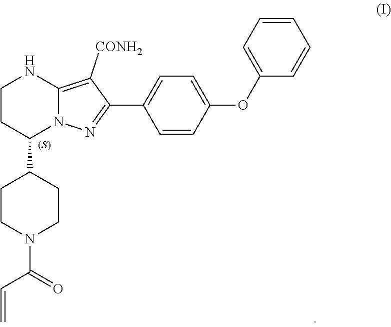

9. A process according to any of claim 8 wherein step (c) comprises:
(i) reacting the compound (III) with compound (IV-A): 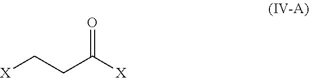
to prepare a compound of formula (II): 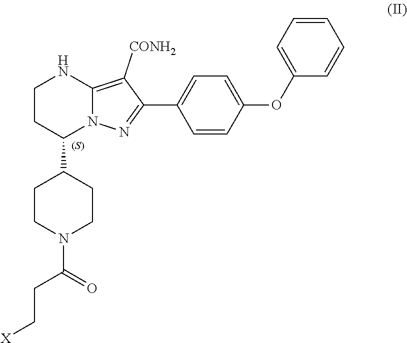
wherein X at either occurrence is the same or different and represents chloro or bromo; and
(ii) elimination to form Zanubrutinib (I).

10. A process according to claim 9, wherein step (i) is carried out in a polar solvent.

11. A process according to claim 9, wherein step (i) is carried out in the presence of a base.

12. A process according to claim 11, wherein the base is selected from an alkali metal carbonate, an alkali metal bicarbonate, or a tertiary C₁₋₄ alkyl amine.

13. A process according to claim 9, wherein the solvent is selected from acetonitrile, tetrahydrofuran, 2-methyl-tetrahydrofuran, methylethylketone, dichloromethane, ethyl acetate, toluene, acetone, or C₁ to C₆ alcohol.

14. A process according to claim 9, wherein step (ii) comprises reacting compound (II) with a base in a solvent.

15. A process according to claim 14, wherein the base is selected from potassium hydroxide, sodium hydroxide, 1,8-Diazabicyclo[5.4.0]undec-7-ene (DBU), t-BuOK, NaOMe, or NaOEt.

16. A process according to claim 14, wherein the solvent is selected from 2 methyltetrahydrofuran (MeTHF), THF, ethyl acetate (EtOAc), acetonitrile, or C₁-C₄ alcohol.

17. A process according to claim 8 wherein step (c) comprises reacting the compound (III) with a compound (IV-B): 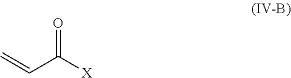
wherein X is chloro or bromo, optionally chloro.

18. A process according to claim 17, wherein the reaction is carried out in the presence of a polar aprotic solvent, in the presence of a base.

19. A process according to claim 17, wherein the base is selected from potassium hydroxide, sodium hydroxide, or 1,8-diazabicyclo[5.4.0]undec-7-ene.

20. A process according to claim 17, wherein the polar aprotic solvent is selected from acetonitrile, tetrahydrofuran, 2-methyl-tetrahydrofuran, water, or mixtures thereof.

21. A process for preparing Zanubrutinib comprising the steps of:
(A) reacting a compound (IX) with a compound (VIII) to form the compound (VII): 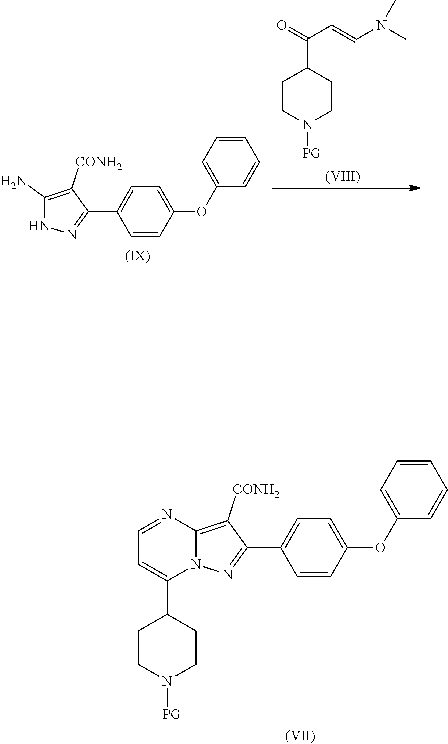
wherein PG is a protecting group;
(B) reducing and deprotecting the compound (VII) to form a compound (VI): 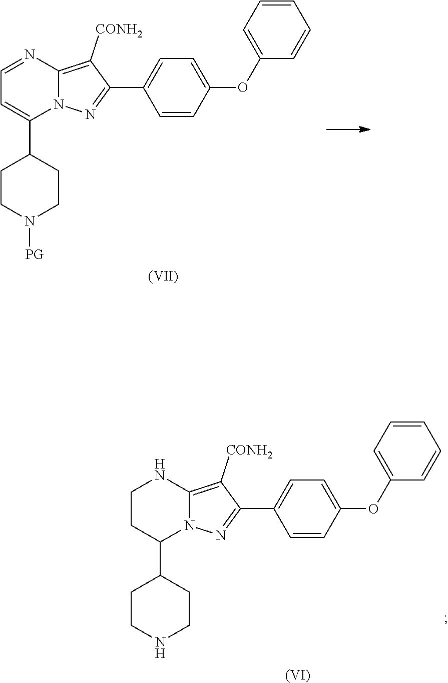
(C) chiral resolution of the compound (VI) to form a compound (III): 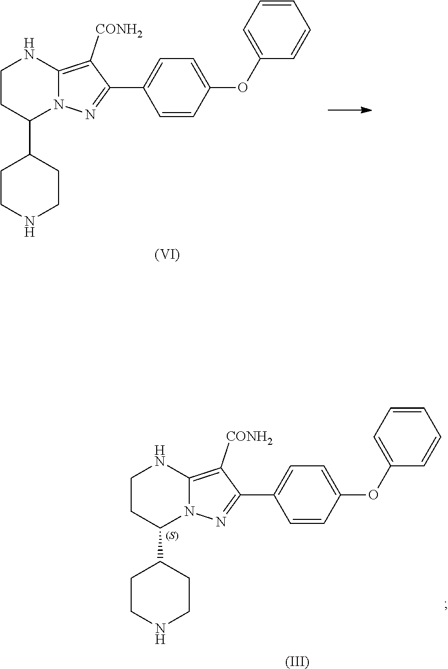
and
(D) N-substitution of the compound (III) 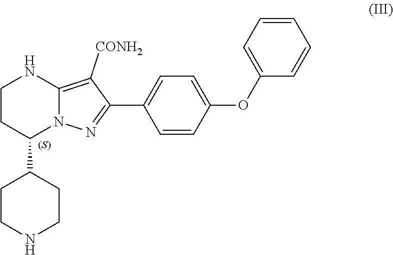
to form Zanubrutinib.

22. A process according to claim 1, further comprising the step of converting Zanubrutinib into a salt thereof.

23. A compound selected from:
Formula (VII): 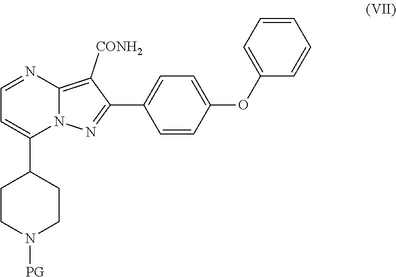
wherein PG is a protecting group; or
Formula (II): 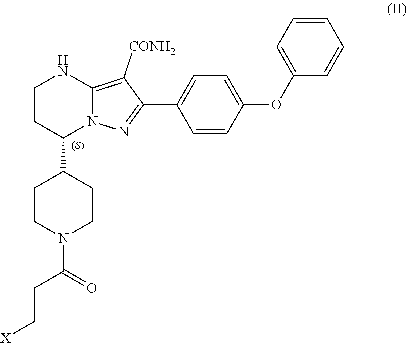
wherein X is chloro or bromo.

24. A process according to claim 1 further comprising combining the Zanubrutinib with at least one pharmaceutically acceptable excipient to form a pharmaceutical formulation.

25. A process according to claim 1, further comprising, sequentially in any order or simultaneously, the steps of reducing and deprotecting the compound (VII), to obtain compound (VI): [chemical structure]
